SEQUENCE LISTING (93 sequences)

SEQ ID NO: 1 - protein, 180 aa, Artificial Sequence
  Synthetic peptide
  MISC_FEATURE (1)..(13): Optional residues
MGSSHHHHHHSSGSMSTDLEKSVERWRELQERLVEEIERLWREALEHSSRSKTESSVEESIKRSLDEIERVIREALERIKELIERSERLYEELDNREDKELGDRALEELLRLQKKLVEDLRRLQEEMNEIARRENSGSGEGLYEALYEKLLDHMVLHERVNAAGITDLYEKLARRMIEEG

SEQ ID NO: 2 - protein, 176 aa, Artificial Sequence
  Synthetic peptide
  MISC_FEATURE (169)..(176): Optional residues
MSMSTDLEKSVERWRELQERLVEEIERLWREALEHSSRSKTESSVEESIKRSLDEIERVIREALERIKELIERSERLYEELDNREDKELGDRAAEELLRLQKKLVEDLRRLQEEMNEIARRENSGSGEGLYEALYKSLMDAALDDLIDTLGGSIDLYEKLSRRMIEEGLEHHHHHH

SEQ ID NO: 3 - protein, 176 aa, Artificial Sequence
  Synthetic peptide
  MISC_FEATURE (169)..(176): Optional residues
MSMSTDLEKSVERWRESQERLVEEIERLWREALEHSSRSKTESSVEESIKRSLDEIERVIREALERIKELIERSERLYEELDNREDKELGDRAAEELLRLQKKLVEDLRRLQEEMNEIARRENSGSGEGLYEALYKSLMDAALDDLIDTLGGSIDLYEKLSRRMIEEGLEHHHHHH

SEQ ID NO: 4 - protein, 176 aa, Artificial Sequence
  Synthetic peptide
  MISC_FEATURE (169)..(176): Optional residues
MSMSTDLEKSVERWRESQERLVEEIERAWREALEHSSRSKTESSVEESIKRSADEIERVIREALERIKELIERSERLYEELDNREDKELGDRAAEELLRLQKKLVEDLRRLQEEMNEIARRENSGSGEGLYEALYKSLMDAALDDLIDTLGGSIDLYEKLSRRMIEEGLEHHHHHH

SEQ ID NO: 5 - protein, 158 aa, Artificial Sequence
  Synthetic peptide
  MISC_FEATURE (1)..(13): Optional residues
GSSHHHHHHSSGSENEELRRIIEEHLRMARRFIENIRRLIEIYEIYEGLGSGSGREEEKSLELSKESIRLLRELLELNRRLLELFRVRESVMELLLDILRLTTRILEEFIKIQEEILDIQRKNTSEEILRRLVEELKKIFELLIRLFELSIDIFRKLE

SEQ ID NO: 6 - protein, 169 aa, Artificial Sequence
  Synthetic peptide
  MISC_FEATURE (1)..(13): Optional residues
GSSHHHHHHSSGSDEEFKKLLDDMSRLLIKMFKEMLDRIIDESRKMWERNNATIEESLDFSKKLWKEIIRILKELSDRILEKLIRELRSVDIDERRLEELLKMLRKLITDIFRDLLEMFERLSEELSRSGSGEGLYAELYEKLIRDLRKDFEKMHSDLLRRLIEKILRI

SEQ ID NO: 7 - protein, 165 aa, Artificial Sequence
  Synthetic peptide
  MISC_FEATURE (1)..(13): Optional residues
GSSHHHHHHSSGLAEELLELLVRISKEVAKILLKAVVKIVEESVRSVKEAEESIRLSVEVWKELIKDLLRVTVKVLKEIAERLRKLNVDHRLLEELLRILIELSKKLLEEALEVIIRLSKELSKVGSGEGLYESLYEELLIRLVRRVIKRHLELLIRVVERVVRV

SEQ ID NO: 8 - protein, 168 aa, Artificial Sequence
  Synthetic peptide
  MISC_FEATURE (1)..(13): Optional residues
GSSHHHHHHSSGSKEEIIRESIEIQRRLVKRIVDELRTTVEELRRMDARIYESTEEINDRILKRVSEVLREALEESRKLLDRARKEVKEKKDTEEVLKEVLELNDRILRKALDDIRKIQERVKRENSGSGSEGLYESLYEKLIEDIIRRLEEAVKESVRLQEELVRKT

SEQ ID NO: 9 - protein, 166 aa, Artificial Sequence
  Synthetic peptide
  MISC_FEATURE (1)..(13): Optional residues
GSSHHHHHHSSGLLEELLRMQEELNKRILEMFHKLLRDLLKMLRELLKDRLPLEELNDHSLHLLRDLLRRILEMSEETLKHILSLWHEIESIEEIFEHLLQLHRRIFDELRKFLDHIQDRLKKLLGSGSEGLYESLYEKLLEEIDKMFKDILEDLLRILDHIFRRM

SEQ ID NO: 10 - protein, 169 aa, Artificial Sequence
  Synthetic peptide
  MISC_FEATURE (1)..(13): Optional residues
GSSHHHHHHSSGSLLERLEELVKHNVDLIRRILELVERAVNIYEIYEGLGSGSDEQEELERLQRELEEVLRRLRENIDELLKLLERTQKLVVTSVLEEILKLIEEQLRILEEALKVLKTSAEVTKRSKELGTREDEEDTLLRLVREILKLVRRLVELVRELLRLAREAT

SEQ ID NO: 11 - protein, 170 aa, Artificial Sequence
  Synthetic peptide
  MISC_FEATURE (1)..(13): Optional residues
GSSHHHHHHSSGSDEEKHEDVVRKLKRLVEELLKLVRKLVEIYEIYEGLGSGSDEQEKSRRMTEELRRMIEEAIRALEEALRLNEKSTVRVSHWAKEEVKRILEELLEVLREALEVLEESLRVQRRSQLHEVNEKDSKELLDRVAKLLERIVERITEIVRRYKELSDRTR

SEQ ID NO: 12 - protein, 167 aa, Artificial Sequence
  Synthetic peptide
  MISC_FEATURE (1)..(13): Optional residues
GSSHHHHHHSSGSLLEELLKIAEDQVRLVDELVKIVDRAVEIYEIYEGLGSGSVNEKAEELQRRTKRILEELKRSAEEIEDLLRKTKKLEVHDLEEKILDVQKKILRLVEEILRLSKRILELTRRSRVRITESLREELVRAVEELVKVVREAVELVRRSVEIVRERT

SEQ ID NO: 13 - protein, 169 aa, Artificial Sequence
  Synthetic peptide
  MISC_FEATURE (1)..(13): Optional residues
GSSHHHHHHSSGDEKEELEKVVRKSRKLIEELLRLVRELLEIYEIYEGLGSGSSASEDLIRINKRVLDLIEEVLESQKELVRLVEESKKHLDKRTEEELIEDVLRKSLRVVERLLELIRRSLEIVKKSTEVLRDSTKEELLEVVREAVRVVEELVKIIRELVRILTETG

SEQ ID NO: 14 - protein, 170 aa, Artificial Sequence
  Synthetic peptide
  MISC_FEATURE (1)..(13): Optional residues
GSSHHHHHHSSGLLEELIKLLKKISEELVRKALKEWVKAVDENAKRVKEEPEDHFVELSVRLSVKMIERVLRQLLEDTVQVLREIVERVVWEIDELKEEALRVLIKISSKLVRELVKLAVKVSKELTKRVSGSEGLYEALYARLISEIVKRALEEHSELLIRIVRELVKV

SEQ ID NO: 15 - protein, 168 aa, Artificial Sequence
  Synthetic peptide
  MISC_FEATURE (1)..(13): Optional residues
GSSHHHHHHSSGREKEEQRKVVKELVRIAREAVDEVRRAVEIYEIYEGLGSGSDKSEEALRVSEELLRKVTELLKMVEKIVDISRKSTDKDTTDRKEDLLRVIEELLRLVRRMVEIVRELVRLSRESTHIVREDSREELVKLVTELVKVAEDLVRVAEEYVKISEEET

SEQ ID NO: 16 - protein, 170 aa, Artificial Sequence
  Synthetic peptide
  MISC_FEATURE (1)..(13): Optional residues
GSSHHHHHHSSGWQDEFSRMFRESSKKLIDIFERMIEEIIDRNEKIILVLHVEKEESLDMSQKLLEEIIELLREMQERILEEIFRAESSHDEKKEEFLEKLRELIERTLKHFLRMYHKIIRELSERIGSGSGSEGLYAELYSELSRRLLEEMMRMNTKLIEELLRELREM

SEQ ID NO: 17 - protein, 166 aa, Artificial Sequence
  Synthetic peptide
  MISC_FEATURE (1)..(13): Optional residues
GSSHHHHHHSSGEERERLLKQVDDTVKRLEEAVKRLREAVNIYEIYEGLGSGDRSEDLLRQTREQLKTLEEVIRKLDESLKTVKKSQKKDTETDVLEKLLEVNDRIKKVIEKLKKVLEESLRVLEKNVNNVEGREKIKEVVRILEELVETLEKLIKKHLDLVRKKT

SEQ ID NO: 18 - protein, 164 aa, Artificial Sequence
  Synthetic peptide
  MISC_FEATURE (1)..(13): Optional residues
GSSHHHHHHSSGASKRLLDLVLEISKRVVENLLKLLEEVVRENAKEVRHRSSEDSIRKSKKALEEVVREVLRQLVEVLERIVREVNVDERLKEEVLRIAIEISERVLREAVKRYIRVSTEMSRRSGSEGLYESLYAELVRRIVKEVLERHSRALMEVVKRVVKL

SEQ ID NO: 19 - protein, 166 aa, Artificial Sequence
  Synthetic peptide
  MISC_FEATURE (1)..(13): Optional residues
GSSHHHHHHSSGKTEEVIRKSIEEIREVVREVVELLRRVVEKNKRTMRDERSKDEAVKRSLETAKRAIDELLKVSKKLIDDLKKTVDISEDADEIITTLLDLNRRAVEELTRVIERIIRELKKATGSGSEGLYEALYERLVRELEKILEDLVRKHVELLKKLRRDQ

SEQ ID NO: 20 - protein, 169 aa, Artificial Sequence
  Synthetic peptide
  MISC_FEATURE (1)..(13): Optional residues
GSSHHHHHHSSGSEEEELLKMARKNFEMIRKMVETVKEAVNIYEIYEGLGSGSDRSEESLRLSEESLRVIREILKLTEEALELIRRTQKKDTDDSVMEELLRVLKEQLEVLKELLEVQEKSLKIQRESSDDRDKDSKELIKDVVEKIERAVRLVKEVVDRSLDIAEKLR

SEQ ID NO: 21 - protein, 165 aa, Artificial Sequence
  Synthetic peptide
  MISC_FEATURE (1)..(13): Optional residues
GSSHHHHHHSSGLAEELLRLLKKSSREVVEKLLRILVELVKENVRQVTEDKMKEKSIRKSVEVLKEVIERVLRLQVKVIEEILRRVVPDLELKEELLRLLIEIVERTVREALRVYIEISVKASEEGSGEGLYESLYLELVERIVREVAKRNTEAVIEIVKRVVKM

SEQ ID NO: 22 - protein, 166 aa, Artificial Sequence
  Synthetic peptide
  MISC_FEATURE (1)..(13): Optional residues
GSSHHHHHHSSGFAEELLRLVAESSERVVRELLKLLLKAVRENVKVATVAEDSIEKSKRVLEKVLEDLLRRQVRMLEEIMRVVIMSDELKKEALEEIIRIIKESVERALEKYIRLSKKMSREVGSGSGSEGLYEALYLKLVREIVKEVVEENLRLLIEIVKEVVKV

SEQ ID NO: 23 - protein, 169 aa, Artificial Sequence
  Synthetic peptide
  MISC_FEATURE (1)..(13): Optional residues
GSSHHHHHHSSGDLEELARESIELLRTIVEEIIRLIRKSADDSKRHKLRRREITETNEEILKRSLDLQVKLLKEVLERIRRVQRDILELVRKEDVKEMLEEVLKRVEEVIRRLLDLSRRIVERLTRENSGSGEGLYESLYEELVKEIVRVLEKIVREYAELQRELIERS

SEQ ID NO: 24 - protein, 168 aa, Artificial Sequence
  Synthetic peptide
  MISC_FEATURE (1)..(13): Optional residues
GSSHHHHHHSSGSMEERLKKLLERQIRLIEELKRLVDRLEEIYEIYEGLGSGSSLIEISEELIRMTEDLFRKLRRLLEESLKLFDDMNDTSGLLELLKELQHRFLRILERLLELQRTSLELQRRSVEHHVPMESIKEILHRIIRIFKELIKILLELSRLFKHIIEHLI

SEQ ID NO: 25 - protein, 160 aa, Artificial Sequence
  Synthetic peptide
  MISC_FEATURE (1)..(13): Optional residues
GSSHHHHHHSSGSLREAVKRSIEIQEDMVRRLKDILKEVADRLTKETDERSSDEINEKSLKDAKRILEEALRELKRLVDEIKKIESKDTEEVLRTVLELNKRLVEELLEDIKRVQEKVKKDGSEGLYESLYERLLEEIIKKLEKVLRESAKLQREAVEKQ

SEQ ID NO: 26 - protein, 162 aa, Artificial Sequence
  Synthetic peptide
  MISC_FEATURE (1)..(13): Optional residues
GSSHHHHHHSSGWLEDIFRIIIKLTEDFLRMLKELLERSLDHNKKNSRPIEESNDTSLKLQEELLDTFLKVQEDLLDKLRRRVVREWLEELIRMFQESMRRLIEIWKEMLTRLLEEFKRRIGSGSEGLYEALYEELLRRLLKLFKDLLRRQKKLLEELLKRW

SEQ ID NO: 27 - protein, 171 aa, Artificial Sequence
  Synthetic peptide
  MISC_FEATURE (1)..(13): Optional residues
GSSHHHHHHSSGSKKELEDLLKRLSEKLEEMLLKLFRDLHKDNKRLVERKEESLEQLKKLQRDLFRHILELTKRLLEELRDRLMKNKVIVDERWIEELIEMLKELSERIFDKFLKMSEKLSEELSRRISGSGSGEGLYAELYENLLERLIREFIKMHLRLLEELIDRIIRI

SEQ ID NO: 28 - protein, 167 aa, Artificial Sequence
  Synthetic peptide
  MISC_FEATURE (1)..(13): Optional residues
GSSHHHHHHSSGWQDELREMFKEISKILLDIFREMIKEILDRNEKLWRHLDKEESKRILEELLREIIRILREISKRLLQRIIEILDEVNVPESSKEEFLKMLEKILEELFRKFLEMYKRLSRKLTDSGSGEGLYSELYEDLIRKLEEKMIRMHTELIERFIDKLLKK

SEQ ID NO: 29 - protein, 175 aa, Artificial Sequence
  Synthetic peptide
  MISC_FEATURE (1)..(13): Optional residues
GSSHHHHHHSSGSKKEMADTSIEIQKELAKRAVEVLEKVVDDLRRTGHRKPEISEDEEEINRDSLKRIKDMLRELLREIERTLDELVRTTRKEGAPEETAKEIVDEVLKLNRKIVRDVLELVREAQERLTKTRGSGSGEGLYESLYEKLVRDIKELLRKVVEDSIRLQRDVVRRT

SEQ ID NO: 30 - protein, 169 aa, Artificial Sequence
  Synthetic peptide
  MISC_FEATURE (1)..(13): Optional residues
GSSHHHHHHSSGKDEDRLVRLAERSERLVEKAEEIVRKLAEIYEIYEGLGSGSDEVETSLEMSRRVIELVKEAIRVVRETNELIRRSQLEIKERSELEELLKINEELLRLLEEWLEIQKEIHRIQKESEERVSEDKKEKVVRVAKELERVVREVVDIARKHAEIVKETR

SEQ ID NO: 31 - protein, 170 aa, Artificial Sequence
  Synthetic peptide
  MISC_FEATURE (1)..(13): Optional residues
GSSHHHHHHSSGDERERNVEVVREAVKTVREVLRQLEDAVEIYEIYEGLGSGSKSEEVLKVTRKNLESIKELLKLLETVKEIQERSRTKNTEDDLLEELVRILDRLEEVVRKLIEIIRRILEIIRRSTHKVVDHRSLEEEAREVVRELERLVRELERIVTEYEKVVRKIG

SEQ ID NO: 32 - protein, 164 aa, Artificial Sequence
  Synthetic peptide
  MISC_FEATURE (1)..(13): Optional residues
GSSHHHHHHSSGAVEELITVVIEASKRVVEELVRKLAEAVERNARRIRHVHKEESVRQLVEIQKRVLRELLKELIKVIKKILEEVVELDEKKEELLRILVKLNDESLREALEMSIRLSKELSKRVGSEGLYESLYEKLLKEVVERVVRENVKLNKEVVERVLRL

SEQ ID NO: 33 - protein, 169 aa, Artificial Sequence
  Synthetic peptide
  MISC_FEATURE (1)..(13): Optional residues
GSSHHHHHHSSGSEDELLQDMLDKSLELIKELLKLIKELVDIYEIYEGLGSGDESEKSEELIKRSLRFLERFEKSQRDFIRILRELIEKVTHESILEILEEILKISKKLLDLWKEIQKESLRIQKEIITVDILDSIREILKRLIKELLRIVEIIVEILKELVRIIKEIV

SEQ ID NO: 34 - protein, 165 aa, Artificial Sequence
  Synthetic peptide
  MISC_FEATURE (1)..(13): Optional residues
GSSHHHHHHSSGDKERAVERWRELQERLVEEIERLWREALEHSSRSKTESSVEESIKRSLDEIERVIREALERIKELIERLKRDADNREDKDEILEELLRLQKKLVEDLRRLQEEMNEIARRENSGSGEGLYEALYEKLLREIVERLRRILKESIDLLKKVVEEG

SEQ ID NO: 35 - protein, 172 aa, Artificial Sequence
  Synthetic peptide
  MISC_FEATURE (1)..(13): Optional residues
GSSHHHHHHSSGFVEEILELLVRTSKRLVERVVEVLVRVIEESVRRLRDLRSEEAVEESLKMSVEVVRRLVEELVREQVKVIKKIADVADVHERLKEEVVRLLIKIIKETAEEIVQEIIKLSVDMSRRVGSGSGSEGLYEALYAKLLKELVDEIVKKNTKALLEVVKRAADV

SEQ ID NO: 36 - protein, 168 aa, Artificial Sequence
  Synthetic peptide
  MISC_FEATURE (1)..(13): Optional residues
GSSHHHHHHSSGSIEELLTEILRITKEMFDELLKLLEEMLRESEKMLDDEEDHRSLEETIRTSLHIFKRMLDEILHLHRRLHEELRKMKSTEEEWLDEMLTDILRSFEELFNDFLRLFEKIHTDLERLSGSEGLYESLYEELLKELKKLLKELLRMQEEMLKELLDRV

SEQ ID NO: 37 - protein, 4 aa, Artificial Sequence
  Synthetic peptide
GGGS

SEQ ID NO: 38 - protein, 8 aa, Artificial Sequence
  Synthetic peptide
GGSGGGGS

SEQ ID NO: 39 - protein, 7 aa, Artificial Sequence
  Synthetic peptide
SGGSGGS

SEQ ID NO: 40 - protein, 15 aa, Artificial Sequence
  Synthetic peptide
GGSGGSGGSGGSGGG

SEQ ID NO: 41 - protein, 16 aa, Artificial Sequence
  Synthetic peptide
GGSGGSGGGGSGGGGS

SEQ ID NO: 42 - protein, 18 aa, Artificial Sequence
  Synthetic peptide
GGSGGSGGSGGSGGSGGS

SEQ ID NO: 43 - protein, 15 aa, Artificial Sequence
  Synthetic peptide
GGGGSGGGGSGGGGS

SEQ ID NO: 44 - protein, 5 aa, Artificial Sequence
  Synthetic peptide
GGGGS

SEQ ID NO: 45 - protein, 15 aa, Artificial Sequence
  Synthetic peptide
GGGGSGGGGSGGGGS

SEQ ID NO: 46 - protein, 8 aa, Artificial Sequence
  Synthetic peptide
  MISC_FEATURE (7)..(7): Xaa is G or S
  MISC_FEATURE (8)..(8): Xaa can be any amino acid except Proline
ENLYFQXX

SEQ ID NO: 47 - protein, 6 aa, Artificial Sequence
  Synthetic peptide
LVPRGS

SEQ ID NO: 48 - protein, 6 aa, Artificial Sequence
  Synthetic peptide
RLVGFE

SEQ ID NO: 49 - protein, 15 aa, Artificial Sequence
  Synthetic peptide
DHMVLHERVNAAGIT

SEQ ID NO: 50 - protein, 16 aa, Artificial Sequence
  Synthetic peptide
GQVGRQLAIIGDDINR

SEQ ID NO: 51 - protein, 16 aa, Artificial Sequence
  Synthetic peptide
GQGGRQMAISGDDNNR

SEQ ID NO: 52 - protein, 14 aa, Artificial Sequence
  Synthetic peptide
MDAALDDLIDTLGG

SEQ ID NO: 53 - protein, 11 aa, Artificial Sequence
  Synthetic peptide
VSGWRLFKKIS

SEQ ID NO: 54 - protein, 16 aa, Artificial Sequence
  Synthetic peptide
RDHMVLHEYVNAAGIT

SEQ ID NO: 55 - protein, 16 aa, Artificial Sequence
  Synthetic peptide
  MISC_FEATURE (2)..(2): Xaa can be any amino acid
  MISC_FEATURE (3)..(3): Xaa can be any amino acid
  MISC_FEATURE (4)..(4): Xaa can be any amino acid
  MISC_FEATURE (7)..(7): Xaa can be any amino acid
  MISC_FEATURE (11)..(11): Xaa can be any amino acid
  MISC_FEATURE (13)..(13): Xaa can be any amino acid
  MISC_FEATURE (14)..(14): Xaa can be any amino acid
  MISC_FEATURE (15)..(15): Xaa can be any amino acid
IXXXLRXIGDXFXXXY

SEQ ID NO: 56 - protein, 20 aa, Artificial Sequence
  Synthetic peptide
EIWIAQELRRIGDEFNAYYA

SEQ ID NO: 57 - protein, 29 aa, Artificial Sequence
  Synthetic peptide
KMAQELIDKVRAASLQINGDAFYAILRAL

SEQ ID NO: 58 - protein, 9 aa, Artificial Sequence
  Synthetic peptide
  MISC_FEATURE (1)..(1): Optional residue
NWSHPQFEK

SEQ ID NO: 59 - protein, 27 aa, Artificial Sequence
  Synthetic peptide
TMFSSNRQKILERTETLNQEWKQRRIQ

SEQ ID NO: 60 - protein, 10 aa, Artificial Sequence
  Synthetic peptide
ETFSDLWKLL

SEQ ID NO: 61 - protein, 33 aa, Artificial Sequence
  Synthetic peptide
GELDELVYLLDGPGYDPIHSDVVTRGGSHLFNF

SEQ ID NO: 62 - protein, 12 aa, Artificial Sequence
  Synthetic peptide
TMDDVYNYLFDD

SEQ ID NO: 63 - protein, 13 aa, Artificial Sequence
  Synthetic peptide
LLTGLFVQYLFDD

SEQ ID NO: 64 - protein, 11 aa, Artificial Sequence
  Synthetic peptide
DDAVVESFFSS

SEQ ID NO: 65 - protein, 9 aa, Artificial Sequence
  Synthetic peptide
GDFLSDLFD

SEQ ID NO: 66 - protein, 9 aa, Artificial Sequence
  Synthetic peptide
GDVLSDLVD

SEQ ID NO: 67 - protein, 13 aa, Artificial Sequence
  Synthetic peptide
NIQMLLEAADYLE

SEQ ID NO: 68 - protein, 13 aa, Artificial Sequence
  Synthetic peptide
NIAMLLAAAAYLE

SEQ ID NO: 69 - protein, 4 aa, Artificial Sequence
  Synthetic peptide
IQIG

SEQ ID NO: 70 - protein, 4 aa, Artificial Sequence
  Synthetic peptide
VQLG

SEQ ID NO: 71 - protein, 11 aa, Artificial Sequence
  Synthetic peptide
VSGWRLFKKIS

SEQ ID NO: 72 - protein, 27 aa, Artificial Sequence
  Synthetic peptide
GLEQEIAALEKENAALEWEIAALEQGG

SEQ ID NO: 73 - protein, 27 aa, Artificial Sequence
  Synthetic peptide
GLKQKIAALKYKNAALKKKIAALKQGG

SEQ ID NO: 74 - protein, 33 aa, Artificial Sequence
  Synthetic peptide
RMKQLEDKVEELLSKNYHLENEVARLKKLVGER

SEQ ID NO: 75 - protein, 30 aa, Artificial Sequence
  Synthetic peptide
GEIAALKQEIAALKKENAALKWEIAALKQG

SEQ ID NO: 76 - protein, 30 aa, Artificial Sequence
  Synthetic peptide
WEAALAEALAEALAEHLAEALAEALEALAA

SEQ ID NO: 77 - protein, 13 aa, Artificial Sequence
  Synthetic peptide
GLFDIIKKIAESF

SEQ ID NO: 78 - protein, 23 aa, Artificial Sequence
  Synthetic peptide
GIGKFLHSAGKFGKAFVGEIMKS

SEQ ID NO: 79 - protein, 23 aa, Artificial Sequence
  Synthetic peptide
GIGKFLHSAKKFGKAFVGEIMNS

SEQ ID NO: 80 - protein, 26 aa, Artificial Sequence
  Synthetic peptide
GIGAVLKVLTTGLPALISWIKRKRQQ

SEQ ID NO: 81 - protein, 14 aa, Artificial Sequence
  Synthetic peptide
INWKGIAAMAKKLL

SEQ ID NO: 82 - protein, 37 aa, Artificial Sequence
  Synthetic peptide
KWKLFKKIEKVGQNIRDGIIKAGPAVAVVGQATQIAK

SEQ ID NO: 83 - protein, 31 aa, Artificial Sequence
  Synthetic peptide
SWLSKTAKKLENSAKKRISEGIAIAIQGGPR

SEQ ID NO: 84 - protein, 16 aa, Artificial Sequence
  Synthetic peptide
GLFDVIKKVASVIGGL

SEQ ID NO: 85 - protein, 14 aa, Artificial Sequence
  Synthetic peptide
NFLGTLINLAKKIL

SEQ ID NO: 86 - protein, 23 aa, Artificial Sequence
  Synthetic peptide
SYFILRRRRKRFPYFFTDVRVAA

SEQ ID NO: 87 - protein, 21 aa, Artificial Sequence
  Synthetic peptide
AFSWGSLWSGIKNFGSTVKNY

SEQ ID NO: 88 - protein, 21 aa, Artificial Sequence
  Synthetic peptide
ASMWERVKSIIKSSLAAASNI

SEQ ID NO: 89 - protein, 50 aa, Artificial Sequence
  Synthetic peptide
VTSTITEKLLKNLIKIISSLVIITRNYEDTTTVLATLALLGCDASPWQWL

SEQ ID NO: 90 - protein, 24 aa, Artificial Sequence
  Synthetic peptide
IAQNPVENYIDEVLNEVLVVPNIN

SEQ ID NO: 91 - protein, 20 aa, Artificial Sequence
  Synthetic peptide
FLGIAEAIDIGNGWEGMEFG

SEQ ID NO: 92 - protein, 20 aa, Artificial Sequence
  Synthetic peptide
GLFGAIAGFIENGWEGMIDG

SEQ ID NO: 93 - protein, 23 aa, Artificial Sequence
  Synthetic peptide
GLFGAIAGFIENGWEGMIDGWYG

(19) United States
(12) Patent Application Publication
(10) Pub. No.: US 2022/0226485 A1
(43) Pub. Date: Jul. 21, 2022

(54) DE NOVO DESIGN OF PHOSPHORYLATION INDUCIBLE PROTEIN SWITCHES (PHOSPHO-SWITCHES)

(71) Applicant: University of Washington, Seattle, WA (US)

(72) Inventors: Nicholas WOODALL, Seattle, WA (US); Scott BOYKEN, Seattle, WA (US); Marc Joseph LAJOIE, Seattle, WA (US); Zibo CHEN, Seattle, WA (US); Robert A. LANGAN, Seattle, WA (US); David BAKER, Seattle, WA (US)

(21) Appl. No.: 17/618,117

(22) PCT Filed: Jun. 17, 2020
(86) PCT No.: PCT/US2020/038048

§ 371 Date: Dec. 10, 2021

Related U.S. Application Data
(60) Provisional application No. 62/964,049, filed on Jan. 21, 2020.
(60) Provisional application No. 62/862,218, filed on Jun. 17, 2019.

(51) Int. Cl. A61K 47/64; C07K 14/00; C12N 15/62; C12N 15/85

(52) U.S. Cl. CPC A61K 47/64; C07K 14/001; A61K 38/00; C12N 15/85; C12N 15/62

(57) ABSTRACT

The present disclosure provides a chimeric polypeptide comprising a helical bundle which comprises between about two and about seven alpha-helices and a bioactive peptide, wherein one or more of the alpha helices form one or more inter-helix hydrogen bonds and comprise at least one phosphorylation site and wherein the bioactive peptide is conformationally placed inside the helical bundle so that the bioactive peptide is not activated or exposed. The disclosure also provides a nucleotide sequence encoding the chimeric polypeptide, a vector comprising the nucleotide sequence, a cell comprising the nucleotide sequence, and method of making, using, or designing the chimeric polypeptide.

### CROSS-REFERENCE TO RELATED APPLICATIONS

This application claims the priority benefit of U.S. Provisional Application No. 62/862,218, filed Jun. 17, 2019, and U.S. Provisional Application No. 62/964,049, filed Jan. 21, 2020, which are herein incorporated by reference in their entirety.

### REFERENCE TO SEQUENCE LISTING SUBMITTED ELECTRONICALLY VIA EFS-WEB

The content of the electronically submitted sequence listing (Name: “19-1079-PCT_Sequence-Listing_ST25.txt” Size: 73 kilobytes; and Date of Creation: Jun. 15, 2020) submitted in this application is incorporated herein by reference in its entirety.

### FIELD OF DISCLOSURE

The present disclosure is directed to a de novo design of a chimeric polypeptide comprising a helical bundle that can change its conformation by an external input, e.g., phosphorylation.

### BACKGROUND

There has been considerable progress in the de novo design of stable protein structures based on the principle that proteins fold into their lowest free energy state. These efforts have focused on maximizing the free energy gap between the desired folded structure and all other structures. Designing proteins that can switch conformations is more challenging, as multiple states must have sufficiently low free energies to be populated relative to the unfolded state, and the free energy differences between the states must be small enough that the state occupancies can be toggled by an external input. The de novo design of a protein system, which switches conformational state in the presence of an external input, e.g., phosphorylation, has not been achieved.

### SUMMARY OF DISCLOSURE

The present disclosure is directed to a chimeric polypeptide comprising a helical bundle comprising between about two and about seven alpha-helices and a bioactive peptide, wherein one or more of the alpha helices form one or more hydrogen bonds and comprise at least one phosphorylation site and wherein the bioactive peptide is conformationally placed inside the helical bundle so that the bioactive peptide is not activated or exposed. In some aspects, one or more of the at least one phosphorylation site is exposed to the exterior surface of the helical bundle. In some aspects, one or more of the at least one phosphorylation site is conformationally buried within the helical bundle such that the phosphorylation site is not exposed. In some aspects, the at least one phosphorylation site is phosphorylated by a kinase (“phosphorylated site”). In some aspects, the phosphorylated site changes the conformation of the helical bundle and exposes one or more phosphorylation sites on the exterior surface of the helical bundle. In some aspects, the phosphorylated site changes the conformation of the helical bundle and exposes or activates the bioactive peptide on the exterior surface of the helical bundle.

The present disclosure also provides a chimeric polypeptide comprising a helical bundle which comprises between about two and about seven alpha-helices and a bioactive peptide, wherein one or more of the alpha helices form one or more inter-helix sidechain hydrogen bonds and comprise at least one phosphorylation site, wherein the phosphorylation site is phosphorylated, and wherein the bioactive peptide is conformationally exposed on the exterior surface of the helical bundle.

In some aspects, the helical bundle useful for the present disclosure comprises at least two, at least three, at least four, or at least five phosphorylation sites. In some aspects, the helical bundle comprises two, three, or four phosphorylation sites. In some aspects, at least two of the phosphorylation site are separated by at least five, at least six, at least seven, at least eight, at least nine, at least ten, at least eleven, at least 12, at least 13, at least 14, at least 15, at least 16, at least 17, at least 18, at least 19, or at least 20 amino acids between the two sites. In some aspects, at least two of the phosphorylation sites are separated by about two to about six amino acid residues between the two sites. In some aspects, the at least two phosphorylation sites are tyrosine residues.

In some aspects, the at least two phosphorylation sites are separated by about two, about three, about four, about five, or about six amino acid residues.

In some aspects, the C-terminal most helical domain of the helical bundle comprises at least one phosphorylation site. In some aspects, the N-terminal most helical domain of the helical bundle comprises at least one phosphorylation site. In some aspects, at least one phosphorylation site is present on the C-terminal helix and at least one phosphorylation site is present on the N-terminal helix. In some aspects, the at least one phosphorylation site at the C-terminal helix is a tyrosine residue and the at least one phosphorylation site at the N-terminal helix is a tyrosine residue.

In some aspects, the at least two phosphorylation sites comprise two phosphorylation sites within 2-3 amino acid residues of each other.

In some aspects, each of the at least two phosphorylation sites comprises a tyrosine residue.

In some aspects, the helical bundle in the chimeric polypeptide further comprises an amino acid linker connecting adjacent alpha helices. In some aspects, the helical bundle comprises two, three, or four alpha helices. In some aspects, one or more of the at least one phosphorylation site is in the C-terminal alpha helix. In some aspects, at least two or three phosphorylation sites are present on the C-terminal alpha helix and at least one phosphorylation site, such as tyrosine, is present on the N-terminal alpha helix.

In some aspects, each helix is independently 30 to 58 amino acids in length.

In some aspects, each of the amino acid linker is independently between 2 and 10 amino acids in length.

In some aspects, the bioactive peptide comprises one or more bioactive peptide selected from Table 2.

In some aspects, one or more of the phosphorylation site are selected from the group consisting of tyrosine, serine, and threonine. In some aspects, the phosphorylation site is tyrosine.

In some aspects, the chimeric polypeptide comprises an amino acid sequence having at least 40%, 45%, 50%, 55%, 60%, 65%, 70%, 75%, 80%, 85%, 90%, 91%, 92%, 93%, 94%, 95%, 96%, 97%, 98%, 99%, or 100% sequence identity along its length to the amino acid sequence selected from the non-limiting group consisting of SEQ ID NOS:1-36. In some aspects, the chimeric polypeptide comprises an amino acid sequence having at least 40%, 45%, 50%, 55%, 60%, 65%, 70%, 75%, 80%, 85%, 90%, 91%, 92%, 93%, 94%, 95%, 96%, 97%, 98%, 99%, or 100% sequence identity along its length to the amino acid sequence selected from the non-limiting group consisting of SEQ ID NOS:1-36. In some aspects, the chimeric polypeptide comprises an amino acid sequence having at least 40%, 45%, 50%, 55%, 60%, 65%, 70%, 75%, 80%, 85%, 90%, 91%, 92%, 93%, 94%, 95%, 96%, 97%, 98%, 99%, or 100% sequence identity along its length to the amino acid sequence selected from the non-limiting group consisting of SEQ ID NOS:1-4, wherein no more than 2, 1, or no phosphorylation sites are present at residues corresponding to residues 1, 3, 4, 7, 8, 11, 14, 15, 18, 19, 22, 26, 29, 30, 33, 36, 37, 39, 40, 41, 42, 45, 46, 49, 53, 56, 57, 60, 64, 67, 68, 71, 75, 78, 79, 81, 82, 83, 84, 86, 87, 90, 91, 94, 98, 101, 102, 105, 108, 109, 112, 113, 116, 120, 123, 124, 126, 127, 128, 132, 135, 136, 139, 143, 146, 147, 150, 154, 157, 158, 161, 165, 166, and 167 of SEQ ID NOS:1-4.

In some aspects, the present disclosure provides a plurality of chimeric polypeptides the chimeric polypeptide of any one of claims 2 and 4 to 31 and the chimeric polypeptide of any one of claims 3 to 31 in equilibrium. In some aspects, a kinase phosphorylates the at least one phosphorylation site on the surface of the helical bundle. In some aspects, the phosphorylated site changes the confirmation of the helical bundle so that one or more phosphorylation sites not exposed on the surface of the helical bundle are exposed on the surface. In some aspects, the phosphorylated sites change the conformation of the helical bundle so that the bioactive peptide is exposed on the surface of the helical bundle.

In some aspects, the disclosure includes a pharmaceutical composition comprising the chimeric polypeptide disclosed herein or the plurality of chimeric polypeptides disclosed herein.

In some aspects, the disclosure comprises a nucleic acid encoding the polypeptide disclosed herein or the plurality of chimeric polypeptides disclosed herein. In some aspects, the disclosure comprises an expression vector comprising the nucleic acid disclosed herein operatively linked to a regulatory sequence. In some aspects, the vector is an adenoviral vector, a lentiviral vector, a baculoviral vector, an Epstein Barr viral vector, a papovaviral vector, a vaccinia viral vector, a herpes simplex viral vector, an adeno associated virus (AAV) vector, or a transposon vector.

In some aspects, the disclosure comprises an in vitro or in vivo cell comprising the nucleic acid disclosed herein or the expression vector disclosed herein. In some aspects, the disclosure provides an ex vivo cell comprising the nucleic acid disclosed herein or the expression vector disclosed herein.

In some aspects, the cell comprises a prokaryotic cell. In some aspects, the cell comprises a yeast cell. In some aspects, the cell comprises a mammalian cell. In some aspects, the mammalian cell comprises HEK 293, CHO, Cos, HeLa, HKB11, or BHK cells.

In some aspects, the cell useful for the present disclosure (e.g., in vitro, in vivo, or ex vivo cells or any host cells) is a human cell. In some aspects, the cell useful for the present disclosure (e.g., in vitro, in vivo, or ex vivo cells or any host cells) is present in a patient or derived from a patient. In some aspects, the patient-derived cell is a tumor cell, cancer cell, immune cell, leukocyte, lymphocyte, T cell, regulatory T cell, effector T cell, CD4+ effector T cell, CD8+ effector T cell, memory T cell, autoreactive T cell, exhausted T cell, natural killer T cell (NKT cells), B cell, dendritic cell, macrophage, NK cell, cardiac cell, lung cell, muscle cell, epithelial cell, pancreatic cell, skin cell, CNS cell, neuron, myocyte, skeletal muscle cell, smooth muscle cell, liver cell, kidney cell, induced pluripotent stem cell (iPSC), embryonic stem cell (ESC), and/or hematopoietic stem cell (HSC). In some aspects, the cell comprises an immune cell. In some aspects, the cell comprises a T cell. In some aspects, the cell comprises a regulatory T cell. In some aspects, the cell comprises a natural killer T cell. In some aspects, the cell comprises an NK cell. In some aspects, the cell comprises an effector T cell, e.g., a CD4+ effector T cell, and/or a CD8+ effector T cell.

In some aspects, the human cell is derived from an allogeneic donor. In some aspects, the allogeneic cell is a tumor cell, cancer cell, immune cell, leukocyte, lymphocyte, T cell, regulatory T cell, effector T cell, CD4+ effector T cell, CD8+ effector T cell, memory T cell, autoreactive T cell, exhausted T cell, natural killer T cell (NKT cells), B cell, dendritic cell, macrophage, NK cell, cardiac cell, lung cell, muscle cell, epithelial cell, pancreatic cell, skin cell, CNS cell, neuron, myocyte, skeletal muscle cell, smooth muscle cell, liver cell, kidney cell, induced pluripotent stem cell (iPSC), embryonic stem cell (ESC), and/or hematopoietic stem cell (HSC).

In some aspects, the cells are engineered to comprise one or more nucleic acids encoding the chimeric polypeptide or to express the chimeric polypeptide described herein. In some aspects, the disclosure provides a host cell comprising the nucleic acid disclosed herein or the expression vector disclosed herein. In some aspects, the nucleic acid or the expression vector is integrated into a host cell chromosome. In some aspects, the nucleic acid or the expression vector is episomal.

In some aspects, the disclosure comprises a method of designing an activatable chimeric polypeptide comprising adding at least one phosphorylation site in a helical bundle, which comprises about two to seven alpha helices and a bioactive peptide, wherein the at least one phosphorylation site is conformationally within the helical bundle such that the phosphorylation site is not exposed. In some aspects, the disclosure comprises a method of designing an activatable chimeric polypeptide comprising adding at least one phosphorylation site in a helical bundle, which comprises about two to seven alpha helices and a bioactive peptide, wherein the at least one phosphorylation site is exposed on the surface of the helical bundle. In some aspects, the disclosure comprises a method of sequestering a bioactive peptide in a chimeric polypeptide comprising adding at least one phosphorylation site in a helical bundle, which comprises about two to seven alpha helices and a bioactive peptide, wherein the at least one phosphorylation site is conformationally within the helical bundle such that the phosphorylation site is not exposed. In some aspects, the at least one phosphorylation site is phosphorylated by a kinase. In some aspects, the method of the present disclosure further comprises phosphorylating the at least one phosphorylation site. In some aspects, the phosphorylating site is selected from the group consisting of tyrosine, serine, or threonine. In some aspects, the phosphorylating site is tyrosine. In some aspects, phosphorylation of the phosphorylating site results in a conformational change that results in phosphorylation of one or more additional phosphorylating sites. In some aspects, phosphorylation of the one or more phosphorylating site results in a conformational change that activates the bioactive peptide.

In some aspects, the disclosure further comprises a method of producing a chimeric polypeptide comprising culturing the host cell disclosed herein under suitable conditions.

### VARIOUS ASPECTS

1. A non-naturally occurring polypeptide comprising a polypeptide comprising a helical bundle, comprising between 2 and 7 alpha-helices, wherein one or more of the alpha helices comprises one or more phosphorylation site.

1A. The polypeptide of Aspect 1, further comprising an amino acid linker connecting adjacent alpha helices.

2. The polypeptide of Aspect 1 or 1A, wherein the alpha helices in total include at least two phosphorylation sites.

3. The polypeptide of any one of Aspects 1-2, wherein the alpha helices in total include at least three phosphorylation sites.

4. The polypeptide of Aspect 2 or 3, wherein the at least two phosphorylation sites comprise two phosphorylation sites within 2-3 amino acid residues of each other, including but not limited to two tyrosine residues separated by 2 or 3 amino acid residues.

5. The polypeptide of any one of Aspects 1-4, wherein the helical bundle comprises 4 alpha helices.

6. The polypeptide of any one of Aspects 1-5, wherein the C-terminal most helical domain comprises at least one phosphorylation site,

6a. The polypeptide of any one of Aspects 1-6, wherein at least three phosphorylation sites, such as tyrosines, are present on the C-terminal helix and at least one phosphorylation site, such as tyrosine, is present on the N-terminal helix.

7. The polypeptide of any one of Aspects 1-6a, wherein each helix is independently 30 to 58 amino acids in length.

8. The polypeptide of any one of Aspects 1A-7, wherein each amino acid linker is independently between 2 and 10 amino acids in length.

9. The polypeptide of any one of Aspects 1-8, wherein the polypeptide comprises a bioactive peptide in at least one of the alpha helices.

10. The polypeptide of Aspect 9, wherein the one or more bioactive peptides may comprise one or more bioactive peptide selected from Table 2.

11. The polypeptide of any one of Aspects 1-10, comprising a polypeptide having at least 40%, 45%, 50%, 55%, 60%, 65%, 70%, 75%, 80%, 85%, 90%, 91%, 92%, 93%, 94%, 95%, 96%, 97%, 98%, 99%, or 100% sequence identity along its length to the amino acid sequence selected from the non-limiting group consisting of SEQ ID NOS:1-36.

12. A non-naturally occurring polypeptide comprising a polypeptide having at least 40%, 45%, 50%, 55%, 60%, 65%, 70%, 75%, 80%, 85%, 90%, 91%, 92%, 93%, 94%, 95%, 96%, 97%, 98%, 99%, or 100% sequence identity along its length to the amino acid sequence selected from the non-limiting group consisting of SEQ ID NOS:1-36.

13. A non-naturally occurring polypeptide comprising a polypeptide having at least 40%, 45%, 50%, 55%, 60%, 65%, 70%, 75%, 80%, 85%, 90%, 91%, 92%, 93%, 94%, 95%, 96%, 97%, 98%, 99%, or 100% sequence identity along its length to the amino acid sequence selected from the non-limiting group consisting of SEQ ID NOS:1-4, wherein no more than 2, 1, or no phosphorylation sites are present at residues corresponding to residues 1,3,4,7,8,11,14,15,18,19,22,26,29,30,33,36,37,39,40,41,42,45,46,49,53,56,57,60,64,67,68,71, 75,78,79,81,82,83,84,86,87,90,91,94,98,101,102,105,108,109,112,113,116,120,123,124,126, 127,128,132,135,136,139,143,146,147,150,154,157,158,161,165,166,167 of SEQ ID NOS:1-4.

14. A nucleic acid encoding the polypeptide of any one of Aspects 1-13.

15. An expression vector comprising the nucleic acid of Aspect 14 operatively linked to a promoter.

16. A host cell comprising the nucleic acid of Aspect 14 or the expression vector of claim 15.

17. The host cell of Aspect 16, wherein the nucleic acid or the expression vector is integrated into a host cell chromosome.

18. The host cell of Aspect 16, wherein the nucleic acid or the expression vector is episomal.

19. Use of the polypeptides, nucleic acids, expression vectors, and/or host cells, disclosed herein to sequester bioactive peptide in the polypeptide, holding them in an inactive (“off”) state, until phosphorylation at the one of more phosphorylation sites induces a conformational change that activates (“on”) the bioactive peptide.

### BRIEF DESCRIPTION OF FIGURES

FIG. 1. Confirmation of the design model. The GFP-11 switch gives the characteristic alpha helical signature by circular dichroism confirming the design model.

FIG. 2. The amount of phosphorylation correlates the activation of GFP fluorescence. Average phosphorylated (total possible 4) was determined by mass spectrometry.

FIG. 3. Activation of DIA switch for binding to the DIV domain of Calpain by biolayer interferometry. “Full binding control”: Full binding to tip control.”+kinase: Phosphorylated DIA switch. “−kinase”: Not phosphorylated DIA switch. “No DIV tip”: No DIV domain negative control.

### DETAILED DESCRIPTION OF DISCLOSURE

The present disclosure is directed to a de novo phosphorylation switch by incorporating hydrogen bond networks containing phosphorylation sites, e.g., tyrosine, serine, or threonine, into a helical bundle. When the key network members, e.g., tyrosines, serines, and/or threonines, become phosphorylated, the very negatively charged phosphate groups destabilize the bundle allowing a caged functional peptide (e.g., bioactive peptide) to perform its bio-active function. The present disclosure includes at least two different switches from this scaffold which activate fluorescence of split-GFP or control binding to the DIV domain of calpain by phosphorylation by the Src family kinases. The designed switches cause up to an 80-fold change in activation after phosphorylation.

All references cited are herein incorporated by reference in their entirety. Within this application, unless otherwise stated, the techniques utilized may be found in any of several well-known references such as: Molecular Cloning: A Laboratory Manual (Sambrook, et al., 1989, Cold Spring Harbor Laboratory Press), Gene Expression Technology (Methods in Enzymology, Vol. 185, edited by D. Goeddel, 1991. Academic Press, San Diego, Calif.), “Guide to Protein Purification” in Methods in Enzymology (M. P. Deutshcer, ed., (1990) Academic Press, Inc.); PCR Protocols: A Guide to Methods and Applications (Innis, et al. 1990. Academic Press, San Diego, Calif.), Culture of Animal Cells: A Manual of Basic Technique, 2^{nd} Ed. (R. I. Freshney. 1987. Liss, Inc. New York, N.Y.), Gene Transfer and Expression Protocols, pp. 109-128, ed. E. J. Murray, The Humana Press Inc., Clifton, N.J.), and the Ambion 1998 Catalog (Ambion, Austin, Tex.).

### I. Definition

As used herein, the singular forms “a”, “an” and “the” include plural referents unless the context clearly dictates otherwise. “And” as used herein is interchangeably used with “or” unless expressly stated otherwise.

As used herein, the amino acid residues are abbreviated as follows: alanine (Ala; A), asparagine (Asn; N), aspartic acid (Asp; D), arginine (Arg; R), cysteine (Cys; C), glutamic acid (Glu; E), glutamine (Gln; Q), glycine (Gly; G), histidine (His; H), isoleucine (Ile; I), leucine (Leu; L), lysine (Lys; K), methionine (Met; M), phenylalanine (Phe; F), proline (Pro; P), serine (Ser; S), threonine (Thr; T), tryptophan (Trp; W), tyrosine (Tyr; Y), and valine (Val; V).

All embodiments of any aspect of the disclosure can be used in combination, unless the context clearly dictates otherwise.

Unless the context clearly requires otherwise, throughout the description and the claims, the words ‘comprise’, ‘comprising’, and the like are to be construed in an inclusive sense as opposed to an exclusive or exhaustive sense; that is to say, in the sense of “including, but not limited to”. Words using the singular or plural number also include the plural and singular number, respectively. Additionally, the words “herein,” “above,” and “below” and words of similar import, when used in this application, shall refer to this application as a whole and not to any particular portions of the application.

Furthermore, “and/or” where used herein is to be taken as specific disclosure of each of the two specified features or components with or without the other. Thus, the term “and/or” as used in a phrase such as “A and/or B” herein is intended to include “A and B,” “A or B,” “A” (alone), and “B” (alone). Likewise, the term “and/or” as used in a phrase such as “A, B, and/or C” is intended to encompass each of the following aspects: A, B, and C; A, B, or C; A or C; A or B; B or C; A and C; A and B; B and C; A (alone); B (alone); and C (alone).

Units, prefixes, and symbols are denoted in their Système International de Unites (SI) accepted form. Numeric ranges are inclusive of the numbers defining the range. Where a range of values is recited, it is to be understood that each intervening integer value, and each fraction thereof, between the recited upper and lower limits of that range is also specifically disclosed, along with each subrange between such values. The upper and lower limits of any range can independently be included in or excluded from the range, and each range where either, neither or both limits are included is also encompassed within the disclosure. Thus, ranges recited herein are understood to be shorthand for all of the values within the range, inclusive of the recited endpoints. For example, a range of 1 to 10 is understood to include any number, combination of numbers, or sub-range from the group consisting of 1, 2, 3, 4, 5, 6, 7, 8, 9, and 10.

Where a value is explicitly recited, it is to be understood that values which are about the same quantity or amount as the recited value are also within the scope of the disclosure. Where a combination is disclosed, each subcombination of the elements of that combination is also specifically disclosed and is within the scope of the disclosure. Conversely, where different elements or groups of elements are individually disclosed, combinations thereof are also disclosed. Where any element of a disclosure is disclosed as having a plurality of alternatives, examples of that disclosure in which each alternative is excluded singly or in any combination with the other alternatives are also hereby disclosed; more than one element of a disclosure can have such exclusions, and all combinations of elements having such exclusions are hereby disclosed.

Nucleotides are referred to by their commonly accepted single-letter codes. Unless otherwise indicated, nucleotide sequences are written left to right in 5′ to 3′ orientation. Nucleotides are referred to herein by their commonly known one-letter symbols recommended by the IUPAC-IUB Biochemical Nomenclature Commission. Accordingly, ‘a’ represents adenine, ‘c’ represents cytosine, ‘g’ represents guanine, ‘t’ represents thymine, and ‘u’ represents uracil.

Amino acid sequences are written left to right in amino to carboxy orientation. Amino acids are referred to herein by either their commonly known three letter symbols or by the one-letter symbols recommended by the IUPAC-IUB Biochemical Nomenclature Commission.

The term “about” is used herein to mean approximately, roughly, around, or in the regions of When the term “about” is used in conjunction with a numerical range, it modifies that range by extending the boundaries above and below the numerical values set forth. In general, the term “about” can modify a numerical value above and below the stated value by a variance of, e.g., 10 percent, up or down (higher or lower).

The term “amino acid substitution” refers to replacing an amino acid residue present in a parent or reference sequence (e.g., a wild type sequence) with another amino acid residue. An amino acid can be substituted in a parent or reference sequence (e.g., a wild type polypeptide sequence), for example, via chemical peptide synthesis or through recombinant methods known in the art. Accordingly, a reference to a “substitution at position X” refers to the substitution of an amino acid present at position X with an alternative amino acid residue. In some aspects, substitution patterns can be described according to the schema AnY, wherein A is the single letter code corresponding to the amino acid naturally or originally present at position n, and Y is the substituting amino acid residue. In other aspects, substitution patterns can be described according to the schema An(YZ), wherein A is the single letter code corresponding to the amino acid residue substituting the amino acid naturally or originally present at position n, and Y and Z are alternative substituting amino acid residues that can replace A.

As used herein, the term “approximately,” as applied to one or more values of interest, refers to a value that is similar to a stated reference value. In certain aspects, the term “approximately” refers to a range of values that fall within 10%, 9%, 8%, 7%, 6%, 5%, 4%, 3%, 2%, 1%, or less in either direction (greater than or less than) of the stated reference value unless otherwise stated or otherwise evident from the context (except where such number would exceed 100% of a possible value).

As used herein, the term “conserved” refers to nucleotides or amino acid residues of a polynucleotide sequence or polypeptide sequence, respectively, that are those that occur unaltered in the same position of two or more sequences being compared. Nucleotides or amino acids that are relatively conserved are those that are conserved amongst more related sequences than nucleotides or amino acids appearing elsewhere in the sequences.

In some aspects, two or more sequences are said to be “completely conserved” or “identical” if they are 100% identical to one another. In some aspects, two or more sequences are said to be “highly conserved” if they are at least about 70% identical, at least about 75% identical, at least about 80% identical, at least about 85% identical, at least about 90% identical, or at least about 95% identical to one another. In some aspects, two or more sequences are said to be “highly conserved” if they are about 70% identical, about 75% identical, about 80% identical, about 85% identical, about 90% identical, about 95% identical, about 98% identical, or about 99% identical to one another. In some aspects, two or more sequences are said to be “conserved” if they are at least about 30% identical, at least about 35% identical, at least about 40% identical, at least about 45% identical, at least about 50% identical, at least about 55%, at least about 60% identical, at least about 65% identical, at least about 70% identical, at least about 75% identical, at least about 80% identical, at least about 85% identical, at least about 90% identical, or at least about 95% identical to one another. In some aspects, two or more sequences are said to be “conserved” if they are about 30% identical, about 35% identical, about 40% identical, about 45% identical, about 50% identical, about 55% identical, about 60% identical, about 65% identical, about 70% identical, about 75% identical, about 80% identical, about 85% identical, about 90% identical, about 95% identical, about 98% identical, or about 99% identical to one another. Conservation of sequence may apply to the entire length of a polynucleotide or polypeptide or may apply to a portion, region or feature thereof.

A “conservative amino acid substitution” is one in which the amino acid residue is replaced with an amino acid residue having a similar side chain. Families of amino acid residues having similar side chains have been defined in the art, including basic side chains (e.g., lysine, arginine, histidine), acidic side chains (e.g., aspartic acid, glutamic acid), uncharged polar side chains (e.g., glycine, asparagine, glutamine, serine, threonine, tyrosine, cysteine), nonpolar side chains (e.g., alanine, valine, leucine, isoleucine, proline, phenylalanine, methionine, tryptophan), beta-branched side chains (e.g., threonine, valine, isoleucine) and aromatic side chains (e.g., tyrosine, phenylalanine, tryptophan, histidine). Thus, if an amino acid in a polypeptide is replaced with another amino acid from the same side chain family, the substitution is considered to be conservative. In another aspect, a string of amino acids can be conservatively replaced with a structurally similar string that differs in order and/or composition of side chain family members.

Non-conservative amino acid substitutions include those in which (i) a residue having an electropositive side chain (e.g., Arg, His or Lys) is substituted for, or by, an electronegative residue (e.g., Glu or Asp), (ii) a hydrophilic residue (e.g., Ser or Thr) is substituted for, or by, a hydrophobic residue (e.g., Ala, Leu, Ile, Phe or Val), (iii) a cysteine or proline is substituted for, or by, any other residue, or (iv) a residue having a bulky hydrophobic or aromatic side chain (e.g., Val, His, Ile or Trp) is substituted for, or by, one having a smaller side chain (e.g., Ala or Ser) or no side chain (e.g., Gly).

Other amino acid substitutions can also be used. For example, for the amino acid alanine, a substitution can be taken from any one of D-alanine, glycine, beta-alanine, L-cysteine and D-cysteine. For lysine, a replacement can be any one of D-lysine, arginine, D-arginine, homo-arginine, methionine, D-methionine, ornithine, or D-ornithine. Generally, substitutions in functionally important regions that can be expected to induce changes in the properties of isolated polypeptides are those in which (i) a polar residue, e.g., serine or threonine, is substituted for (or by) a hydrophobic residue, e.g., leucine, isoleucine, phenylalanine, or alanine; (ii) a cysteine residue is substituted for (or by) any other residue; (iii) a residue having an electropositive side chain, e.g., lysine, arginine or histidine, is substituted for (or by) a residue having an electronegative side chain, e.g., glutamic acid or aspartic acid; or (iv) a residue having a bulky side chain, e.g., phenylalanine, is substituted for (or by) one not having such a side chain, e.g., glycine. The likelihood that one of the foregoing non-conservative substitutions can alter functional properties of the protein is also correlated to the position of the substitution with respect to functionally important regions of the protein: some non-conservative substitutions can accordingly have little or no effect on biological properties.

In the content of the present disclosure, the terms “mutation” and “amino acid substitution” as defined above (sometimes referred simply as a “substitution”) are considered interchangeable.

In the context of the present disclosure, substitutions (even when they are referred to as amino acid substitution) are conducted at the nucleic acid level, i.e., substituting an amino acid residue with an alternative amino acid residue is conducted by substituting the codon encoding the first amino acid with a codon encoding the second amino acid.

As used herein, the term “homology” refers to the overall relatedness between polymeric molecules, e.g. between nucleic acid molecules (e.g. DNA molecules and/or RNA molecules) and/or between polypeptide molecules. Generally, the term “homology” implies an evolutionary relationship between two molecules. Thus, two molecules that are homologous will have a common evolutionary ancestor. In the context of the present disclosure, the term homology encompasses both to identity and similarity.

In some aspects, polymeric molecules are considered to be “homologous” to one another if at least about 25%, at least about 30%, at least about 35%, at least about 40%, at least about 45%, at least about 50%, at least about 55%, at least about 60%, at least about 65%, at least about 70%, at least about 75%, at least about 80%, at least about 85%, at least about 90%, at least about 95%, or at least about 99% of the monomers in the molecule are identical (exactly the same monomer) or are similar (conservative substitutions). The term “homologous” necessarily refers to a comparison between at least two sequences (polynucleotide or polypeptide sequences).

As used herein, the term “identity” refers to the overall monomer conservation between polymeric molecules, e.g., between polypeptide molecules or polynucleotide molecules (e.g. DNA molecules and/or RNA molecules). The term “identical” without any additional qualifiers, e.g., protein A is identical to protein B, implies the sequences are 100% identical (100% sequence identity). Describing two sequences as, e.g., “70% identical,” is equivalent to describing them as having, e.g., “70% sequence identity.”

Calculation of the percent identity of two polypeptide sequences, for example, can be performed by aligning the two sequences for optimal comparison purposes (e.g., gaps can be introduced in one or both of a first and a second polypeptide sequences for optimal alignment and non-identical sequences can be disregarded for comparison purposes). In certain aspects, the length of a sequence aligned for comparison purposes is at least 30%, at least 40%, at least 50%, at least 60%, at least 70%, at least 80%, at least 90%, at least 95%, or 100% of the length of the reference sequence. The amino acids at corresponding amino acid positions are then compared.

When a position in the first sequence is occupied by the same amino acid as the corresponding position in the second sequence, then the molecules are identical at that position. The percent identity between the two sequences is a function of the number of identical positions shared by the sequences, taking into account the number of gaps, and the length of each gap, which needs to be introduced for optimal alignment of the two sequences. The comparison of sequences and determination of percent identity between two sequences can be accomplished using a mathematical algorithm.

Suitable software programs are available from various sources, and for alignment of both protein and nucleotide sequences. One suitable program to determine percent sequence identity is bl2seq, part of the BLAST suite of program available from the U.S. government's National Center for Biotechnology Information BLAST web site (blast.ncbi.nlm.nih.gov). Bl2seq performs a comparison between two sequences using either the BLASTN or BLASTP algorithm. BLASTN is used to compare nucleic acid sequences, while BLASTP is used to compare amino acid sequences. Other suitable programs are, e.g., Needle, Stretcher, Water, or Matcher, part of the EMBOSS suite of bioinformatics programs and also available from the European Bioinformatics Institute (EBI) at web site ebi.ac.uk/Tools/psa. Sequence alignments can be conducted using methods known in the art such as MAFFT, Clustal (ClustalW, Clustal X or Clustal Omega), MUSCLE, etc. Different regions within a single polynucleotide or polypeptide target sequence that aligns with a polynucleotide or polypeptide reference sequence can each have their own percent sequence identity. It is noted that the percent sequence identity value is rounded to the nearest tenth. For example, 80.11, 80.12, 80.13, and 80.14 are rounded down to 80.1, while 80.15, 80.16, 80.17, 80.18, and 80.19 are rounded up to 80.2. It also is noted that the length value will always be an integer.

In certain aspects, the percentage identity (% ID) or of a first amino acid sequence (or nucleic acid sequence) to a second amino acid sequence (or nucleic acid sequence) is calculated as % ID=100×(Y/Z), where Y is the number of amino acid residues (or nucleobases) scored as identical matches in the alignment of the first and second sequences (as aligned by visual inspection or a particular sequence alignment program) and Z is the total number of residues in the second sequence. If the length of a first sequence is longer than the second sequence, the percent identity of the first sequence to the second sequence will be higher than the percent identity of the second sequence to the first sequence.

One skilled in the art will appreciate that the generation of a sequence alignment for the calculation of a percent sequence identity is not limited to binary sequence-sequence comparisons exclusively driven by primary sequence data. It will also be appreciated that sequence alignments can be generated by integrating sequence data with data from heterogeneous sources such as structural data (e.g., crystallographic protein structures), functional data (e.g., location of mutations), or phylogenetic data. A suitable program that integrates heterogeneous data to generate a multiple sequence alignment is T-Coffee, available at www.tcoffee.org, and alternatively available, e.g., from the EBI. It will also be appreciated that the final alignment used to calculate percent sequence identity can be curated either automatically or manually.

As used herein, the term “similarity” refers to the overall relatedness between polymeric molecules, e.g. between polynucleotide molecules (e.g. DNA molecules and/or RNA molecules) and/or between polypeptide molecules. Calculation of percent similarity of polymeric molecules to one another can be performed in the same manner as a calculation of percent identity, except that calculation of percent similarity takes into account conservative substitutions as is understood in the art. It is understood that percentage of similarity is contingent on the comparison scale used, i.e., whether the amino acids are compared, e.g., according to their evolutionary proximity, charge, volume, flexibility, polarity, hydrophobicity, aromaticity, isoelectric point, antigenicity, or combinations thereof.

“Nucleic acid,” “nucleic acid molecule,” “nucleotide sequence,” “polynucleotide,” and grammatical variants thereof are used interchangeably and refer to the phosphate ester polymeric form of ribonucleosides (adenosine, guanosine, uridine or cytidine; “RNA molecules”) or deoxyribonucleosides (deoxyadenosine, deoxyguanosine, deoxythymidine, or deoxycytidine; “DNA molecules”), or any phosphoester analogs thereof, such as phosphorothioates and thioesters, in either single stranded form, or a double-stranded helix. Single stranded nucleic acid sequences refer to single-stranded DNA (ssDNA) or single-stranded RNA (ssRNA). Double stranded DNA-DNA, DNA-RNA and RNA-RNA helices are possible. The term nucleic acid molecule, and in particular DNA or RNA molecule, refers only to the primary and secondary structure of the molecule, and does not limit it to any particular tertiary forms. Thus, this term includes double-stranded DNA found, inter alia, in linear or circular DNA molecules (e.g., restriction fragments), plasmids, supercoiled DNA and chromosomes. In discussing the structure of particular double-stranded DNA molecules, sequences can be described herein according to the normal convention of giving only the sequence in the 5′ to 3′ direction along the non-transcribed strand of DNA (i.e., the strand having a sequence homologous to the mRNA).

The term “polynucleotide” as used herein refers to polymers of nucleotides of any length, including ribonucleotides, deoxyribonucleotides, analogs thereof, or mixtures thereof. This term refers to the primary structure of the molecule. Thus, the term includes triple-, double- and single-stranded deoxyribonucleic acid (“DNA”), as well as triple-, double- and single-stranded ribonucleic acid (“RNA”). It also includes modified, for example by alkylation, and/or by capping, and unmodified forms of the polynucleotide. More particularly, the term “polynucleotide” includes polydeoxyribonucleotides (containing 2-deoxy-D-ribose) and polyribonucleotides (containing D-ribose), including mRNA, whether spliced or unspliced, any other type of polynucleotide which is an N- or C-glycoside of a purine or pyrimidine base, and other polymers containing normucleotidic backbones, for example, polyamide (e.g., peptide nucleic acids “PNAs”) and polymorpholino polymers, and other synthetic sequence-specific nucleic acid polymers providing that the polymers contain nucleobases in a configuration which allows for base pairing and base stacking, such as is found in DNA and RNA.

In some aspects, a polynucleotide disclosed herein comprises a DNA, e.g., a DNA inserted in a vector. In other aspects, a polynucleotide disclosed herein comprises an mRNA. In some aspects, the mRNA is a synthetic mRNA. In some aspects, the synthetic mRNA comprises at least one unnatural nucleobase. In some aspects, all nucleobases of a certain class have been replaced with unnatural nucleobases (e.g., all uridines in a polynucleotide disclosed herein can be replaced with an unnatural nucleobase, e.g., 5-methoxyuridine).

The term “encoding” refers to the inherent property of specific sequences of nucleotides in a polynucleotide, such as a gene, a cDNA, or an mRNA, to serve as templates for synthesis of other polymers and macromolecules in biological processes having either a defined sequence of nucleotides (e.g., rRNA, tRNA and mRNA) or a defined sequence of amino acids and the biological properties resulting therefrom. Thus, a gene, cDNA, or RNA, encodes a protein if transcription and translation of mRNA corresponding to that gene produces the protein in a cell or other biological system. Both the coding strand, the nucleotide sequence of which is identical to the mRNA sequence and is usually provided in sequence listings, and the non-coding strand, used as the template for transcription of a gene or cDNA, can be referred to as encoding the protein or other product of that gene or cDNA.

Unless otherwise specified, a nucleotide sequence “encoding” an amino acid sequence,” e.g., a polynucleotide “encoding” a chimeric polypeptide of the present disclosure, includes all nucleotide sequences that are degenerate versions of each other and that encode the same amino acid sequence.

The term “expression” refers to the transcription and/or translation of a particular nucleotide sequence driven by a promoter.

As used throughout the present application, the term “polypeptide” is used in its broadest sense to refer to a sequence of subunit amino acids. The polypeptides of the disclosure can comprise L-amino acids+glycine, D-amino acids+glycine (which are resistant to L-amino acid-specific proteases in vivo), or a combination of D- and L-amino acids+glycine. The polypeptides described herein can be chemically synthesized or recombinantly expressed.

The polypeptides of the disclosure can include additional residues at the N-terminus, C-terminus, internal to the polypeptide, or a combination thereof; these additional residues are not included in determining the percent identity of the polypeptides of the disclosure relative to the reference polypeptide. Such residues may be any residues suitable for an intended use, including but not limited to tags. As used herein, “tags” include general detectable moieties (i.e.: fluorescent proteins, antibody epitope tags, etc.), therapeutic agents, purification tags (His tags, etc.), linkers, ligands suitable for purposes of purification, ligands to drive localization of the polypeptide, peptide domains that add functionality to the polypeptides, etc.

The terms “polypeptide,” “peptide,” and “protein” are used interchangeably herein to refer to polymers of amino acids of any length. The polymer can comprise modified amino acids. The terms also encompass an amino acid polymer that has been modified naturally or by intervention; for example, disulfide bond formation, glycosylation, lipidation, acetylation, phosphorylation, or any other manipulation or modification, such as conjugation with a labeling component. Also included within the definition are, for example, polypeptides containing one or more analogs of an amino acid (including, for example, unnatural amino acids such as homocysteine, ornithine, p-acetylphenylalanine, D-amino acids, and creatine), as well as other modifications known in the art.

The term “polypeptide,” as used herein, refers to proteins, polypeptides, and peptides of any size, structure, or function. Polypeptides include gene products, naturally occurring polypeptides, synthetic polypeptides, homologs, orthologs, paralogs, fragments and other equivalents, variants, and analogs of the foregoing. A polypeptide can be a single polypeptide or can be a multi-molecular complex such as a dimer, trimer or tetramer. They can also comprise single chain or multichain polypeptides. Most commonly disulfide linkages are found in multichain polypeptides. The term polypeptide can also apply to amino acid polymers in which one or more amino acid residues are an artificial chemical analogue of a corresponding naturally occurring amino acid. In some aspects, a “peptide” can be less than or equal to 50 amino acids long, e.g., about 5, 10, 15, 20, 25, 30, 35, 40, 45, or 50 amino acids long.

The term “non-naturally occurring” is used herein to mean a polypeptide or a polynucleotide sequence that does not exist in nature. In some aspects, the non-naturally occurring sequence does not exist in nature because it is a combination of two known, naturally-occurring, sequences (e.g., chimeric polypeptide) that do not occur together in nature. In some aspects, a non-naturally occurring polypeptide is a chimeric polypeptide. In some aspects, a polypeptide or a polynucleotide is not naturally occurring because the sequence contains a portion (e.g., a fragment) that cannot be found in nature, i.e., a novel sequence.

A “chimeric polypeptide” as used herein, refers to any polypeptide comprised of a first amino acid sequence derived from a first source, bonded, covalently or non-covalently, to a second amino acid sequence derived from a second source, wherein the first and second source are not the same. A first source and a second source that are not the same can include two different biological entities, or two different proteins from the same biological entity, or a biological entity and a non-biological entity. A chimeric protein can include for example, a protein derived from at least 2 different biological sources. A biological source can include any non-synthetically produced nucleic acid or amino acid sequence (e.g. a genomic or cDNA sequence, a plasmid or viral vector, a native virion or a mutant or analog, as further described herein, of any of the above). A synthetic source can include a protein or nucleic acid sequence produced chemically and not by a biological system (e.g. solid phase synthesis of amino acid sequences). A chimeric protein can also include a protein derived from at least 2 different synthetic sources or a protein derived from at least one biological source and at least one synthetic source. A chimeric protein may also comprise a first amino acid sequence derived from a first source, covalently or non-covalently linked to a nucleic acid, derived from any source or a small organic or inorganic molecule derived from any source. The chimeric protein can comprise a linker molecule between the first and second amino acid sequence or between the first amino acid sequence and the nucleic acid, or between the first amino acid sequence and the small organic or inorganic molecule.

As used herein, the term “fragment” of a polypeptide refers to an amino acid sequence of a polypeptide that is shorter than the naturally-occurring sequence, N- and/or C-terminally deleted or any part of the polypeptide deleted in comparison to the naturally occurring polypeptide. Thus, a fragment does not necessary need to have only N- and/or C- terminal amino acids deleted. A polypeptide in which internal amino acids have been deleted with respect to the naturally occurring sequence is also considered a fragment.

As used herein, the term “functional fragment” refers to a polypeptide fragment that retains polypeptide function. Accordingly, in some aspects, a functional fragment of a bioactive peptide, e.g., an enzyme, retains the ability to catalyze a biological action, e.g., having a catalytic domain of the enzyme.

As used herein, a “phosphorylation site” is any amino acid residue or motif of residues that can be phosphorylated by a kinase. In various embodiments, the phosphorylation site may be a tyrosine residue, a serine phosphorylation motif, a threonine phosphorylation motif, or combinations thereof. In the examples that follow, the phosphorylation sites comprise tyrosine residues, but those of skill in the art will understand, based on the teachings herein, that any suitable serine or threonine phosphorylation motif can be substituted for the tyrosine residue, or may be included in addition to the tyrosine residue. As will also be apparent to those of skill in the art based on the teachings herein, the position of the phosphorylation site is not limited by the examples shown below.

The term “phosphorylated sites” as used herein means one or more amino acids, e.g., tyrosine, serine, and/or threonine, that have been phosphorylated.

The description of embodiments of the disclosure is not intended to be exhaustive or to limit the disclosure to the precise form disclosed. While the specific embodiments of, and examples for, the disclosure are described herein for illustrative purposes, various equivalent modifications are possible within the scope of the disclosure, as those skilled in the relevant art will recognize.

### II. Phospho-Switch Polypeptides

As described herein, the non-naturally occurring polypeptides disclosed herein can be used as cage polypeptides that can, for example, sequester a bioactive peptide in an inactive state until phosphorylation at the one or more phosphorylation sites activates the bioactive peptide. The helical bundle useful for the disclosure comprises the exterior surface and the internal space. Any sites (or residues) exposed on the exterior surface of the bundle can come into a contact with a biological moiety or can be a target (e.g., epitope) for a biological moiety. Some residues (or a sequence) in the helical bundle can be buried within the internal space and cannot be exposed to the surface. When phosphorylation sites (i.e., residues) are buried within the internal space, the sites cannot be phosphorylated. When the residues in a bioactive peptide that are necessary for activation are buried within the internal space, the bioactive peptide is not activated. In some embodiments, the phosphorylation sites are at helical residue positions.

In some aspects, the disclosure comprises a chimeric polypeptide comprising a helical bundle which comprises between about two and about seven alpha-helices and a bioactive peptide, wherein one or more of the alpha helices form one or more hydrogen bonds and comprise at least one phosphorylation site and wherein the bioactive peptide is conformationally placed inside the helical bundle so that the bioactive peptide is not activated or exposed. In some aspects, one or more of the at least one phosphorylation site is exposed to the exterior surface of the helical bundle. In some aspects, one or more of the at least one phosphorylation site is conformationally buried within the helical bundle such that the phosphorylation site is not exposed. In some aspects, the phosphorylation site is selected from tyrosine, serine, or threonine. In some aspects, the phosphorylation site is tyrosine. In some aspects, the phosphorylation site is serine. In some aspects, the phosphorylation site is threonine.

The one or more phosphorylation site may be any residue that when phosphorylated causes a decrease in the stability of the protein's folded state or unphosphorylated conformation. From a structural perspective, the decrease in stability may occur from any structured residue, current rotamer or possibly sampled rotamers, that the addition of the negatively charged phosphate group would cause a steric clash from the bulk of the phosphate group with any other residues, electronic repulsion from the negative charge from any other residues, or positioning within a hydrophobic section of the protein from the hydrophobic effect. In one embodiment, no more than two, one, or no phosphorylation sites are present on an exterior surface of the polypeptide. The polypeptides are designed to keep the phosphorylation sites buried in the designed state, but to have just enough dynamics/breathability of the polypeptide scaffold such that these phosphorylation sites become transiently/infrequently exposed, just enough to get phosphorylated by kinase and activate the switch. In some embodiments, “destabilizing mutations” are added (as exemplified below) to weaken the scaffold and increase this breathing/accessibility).

In some aspects, the at least one phosphorylation site in a helical bundle is phosphorylated by a kinase (“phosphorylated site”). The phosphorylated site in turn can change the conformation of the helical bundle and allow one or more additional phosphorylation sites that were conformationally buried within the helical bundle to be exposed on the surface of the helical bundle. Therefore, the first phosphorylated site can further expose the second phosphorylation site, thereby allowing the second phosphorylation site to be phosphorylated. The conformational changes due to the phosphorylation of the amino acid sites further induce conformational changes such that the bioactive peptide previously buried within the helical bundle is activated or exposed on the surface of the helical bundle.

In some aspects, the disclosure is directed to a chimeric polypeptide comprising a helical bundle comprising between about two and about seven alpha-helices and a bioactive peptide, wherein one or more of the alpha helices form one or more hydrogen bonds and comprise at least one phosphorylation site, wherein the phosphorylation site is phosphorylated, and wherein the bioactive peptide is conformationally exposed on the surface of the helical bundle.

In some aspects, the helical bundle comprises at least two, at least three, at least four, or at least five phosphorylation sites. In some aspects, the helical bundle comprises two phosphorylation sites. In some aspects, the helical bundle comprises three phosphorylation sites. In some aspects, the helical bundle comprises four phosphorylation sites. In some aspects, the helical bundle comprises five phosphorylation sites. In some aspects, at least two of the phosphorylation sites are separated by at least five, at least six, at least seven, at least eight, at least nine, at least ten, at least eleven, at least 12, at least 13, at least 14, at least 15, at least 16, at least 17, at least 18, at least 19, or at least 20 amino acids between the two sites. In some aspects, at least two of the phosphorylation sites, e.g., tyrosine residues, are separated by at least five amino acids between the two sites, e.g., Y₁X₁X₂X₃X₄X₅Y₂. In some aspects, at least two of the phosphorylation sites, e.g., tyrosine residues, are separated by at least six amino acids between the two sites. In some aspects, at least two of the phosphorylation sites, e.g., tyrosine residues, are separated by at least seven amino acids between the two sites. In some aspects, at least two of the phosphorylation sites, e.g., tyrosine residues, are separated by at least eight amino acids between the two sites. In some aspects, at least two of the phosphorylation sites, e.g., tyrosine residues, are separated by at least nine amino acids between the two sites. In some aspects, at least two of the phosphorylation sites, e.g., tyrosine residues, are separated by at least 10 amino acids between the two sites. In some aspects, at least two of the phosphorylation sites, e.g., tyrosine residues, are separated by at least 11 amino acids between the two sites. In some aspects, at least two of the phosphorylation sites, e.g., tyrosine residues, are separated by at least 12 amino acids between the two sites. In some aspects, at least two of the phosphorylation sites, e.g., tyrosine residues, are separated by at least 13 amino acids between the two sites. In some aspects, at least two of the phosphorylation sites, e.g., tyrosine residues, are separated by at least 14 amino acids between the two sites. In some aspects, at least two of the phosphorylation sites, e.g., tyrosine residues, are separated by at least 15 amino acids between the two sites. In some aspects, at least two of the phosphorylation sites, e.g., tyrosine residues, are separated by at least 16 amino acids between the two sites. In some aspects, at least two of the phosphorylation sites, e.g., tyrosine residues, are separated by at least 17 amino acids between the two sites. In some aspects, at least two of the phosphorylation sites, e.g., tyrosine residues, are separated by at least 18 amino acids between the two sites. In some aspects, at least two of the phosphorylation sites, e.g., tyrosine residues, are separated by at least 19 amino acids between the two sites. In some aspects, at least two of the phosphorylation sites, e.g., tyrosine residues, are separated by at least 20 amino acids between the two sites.

In some aspects, the at least two phosphorylation site comprises two phosphorylation sites within 2-3 amino acid residues of each other, including but not limited to two tyrosine residues separated by 2 or 3 amino acid residues.

In some aspects, at least two phosphorylation sites, e.g., tyrosine residues, are separated by about two to about six amino acid residues between the two sites, e.g., Y₁X₁X₂Y₂. In some aspects, the two phosphorylation sites, e.g., tyrosine residues, are separated by about two amino acid residues between the two sites. In some aspects, the two phosphorylation sites, e.g., tyrosine residues, are separated by about three amino acid residues between the two sites. In some aspects, the two phosphorylation sites, e.g., tyrosine residues, are separated by about four amino acid residues between the two sites. In some aspects, the two phosphorylation sites, e.g., tyrosine residues, are separated by about five amino acid residues between the two sites. In some aspects, the two phosphorylation sites, e.g., tyrosine residues, are separated by about six amino acid residues between the two sites.

In some aspects, at least two phosphorylation sites, e.g., tyrosine residues, are separated by one amino acid residue between the two sites, e.g., Y₁XY₂.

In some aspects, the C-terminal most helical domain of the helical bundle comprises at least one phosphorylation site, e.g., tyrosine. In some aspects, the N-terminal most helical domain of the helical bundle comprises at least one phosphorylation site, e.g., tyrosine. In some aspects, at least one phosphorylation site is present on the C-terminal helix and at least one phosphorylation site is present on the N-terminal helix. In some aspects, the at least one phosphorylation site at the C-terminal helix is a tyrosine residue and the at least one phosphorylation site at the N-terminal helix is a tyrosine residue.

In some aspects, the first of the two phosphorylation sites is threonine or serine, and the second is tyrosine. In some aspects, the first of the two phosphorylation sites is threonine and the second is tyrosine. In some aspects, the first of the two phosphorylation sites is serine and the second is tyrosine. In some aspects, the first of the two phosphorylation sites is tyrosine and the second is tyrosine.

In some aspects, the helical bundle comprises three phosphorylation sites; for example, the first is tyrosine, the second is tyrosine and the third is tyrosine. In some aspects, the first is tyrosine, the second is serine or threonine, and the third is tyrosine. In some aspects, the first is tyrosine, the second is serine or threonine, and the third is serine or threonine. In some aspects, the first is serine or threonine, the second is serine or threonine, and the third is serine or threonine.

In some aspects, the helical bundle comprises four phosphorylation sites; for example, the first is tyrosine, the second is tyrosine, the third is tyrosine; and the fourth is tyrosine. In some aspects, the first is tyrosine, the second is serine or threonine, the third is tyrosine, and the fourth is tyrosine. In some aspects, the first is tyrosine, the second is serine or threonine, the third is serine or threonine, and the fourth is tyrosine. In some aspects, the first is serine or threonine, the second is serine or threonine, the third is serine or threonine, and the fourth is tyrosine. In some aspects, the first is serine or threonine, the second is serine or threonine, the third is serine or threonine, and the fourth is serine or threonine. In some aspects, all phosphorylation sites are serine. In some aspects, all phosphorylation sites are threonine.

In some aspects, one of all phosphorylation sites in the helical bundle is tyrosine. In some aspects, two of all phosphorylation sites in the helical bundle are tyrosine. In some aspects, three of all phosphorylation sites in the helical bundle are tyrosine. In some aspects, four of all phosphorylation sites in the helical bundle are tyrosine.

In some aspects, one of all phosphorylation sites in the helical bundle is serine. In some aspects, two of all phosphorylation sites in the helical bundle are serine. In some aspects, three of all phosphorylation sites in the helical bundle are serine. In some aspects, four of all phosphorylation sites in the helical bundle are serine.

In some aspects, one of all phosphorylation sites in the helical bundle is threonine. In some aspects, two of all phosphorylation sites in the helical bundle are threonine. In some aspects, three of all phosphorylation sites in the helical bundle are threonine. In some aspects, four of all phosphorylation sites in the helical bundle are threonine.

In some aspects, the helical bundle of the present disclosure comprises two, three, four, five, six, or seven alpha helices. In some aspects, the helical bundle comprises two alpha helices. In some aspects, the helical bundle comprises three alpha helices. In some aspects, the helical bundle comprises four alpha helices. In some aspects, the helical bundle comprises five alpha helices. In some aspects, the helical bundle comprises six alpha helices. In some aspects, the helical bundle comprises seven alpha helices. In some aspects, one or more of the at least one phosphorylation site is in the C-terminal alpha helix. In some aspects, at least two or three phosphorylation sites are present on the C-terminal alpha helix and at least one phosphorylation site, such as tyrosine, is present on the N-terminal alpha helix. In some aspects, the helical bundle comprises one or more linkers. In some aspects, a linker for the helical bundle can connect two adjacent alpha helices.

In some aspects, a helical bundle of the disclosure comprises a bioactive peptide. In some aspects, a helical bundle of the disclosure comprises a linker connecting the bioactive peptide and an alpha helix. In some aspects, the bioactive peptide useful for the present disclosure can be selected from Table 2.

In some aspects, the present disclosure also provides a plurality of chimeric polypeptides comprising a chimeric polypeptide which comprises a helical bundle comprising between about two and about seven alpha-helices and a bioactive peptide, wherein one or more of the alpha helices form one or more hydrogen bonds and comprise at least one phosphorylation site and wherein the bioactive peptide is conformationally placed inside the helical bundle so that the bioactive is not activated or exposed. In the plurality of chimeric polypeptides, one or more chimeric polypeptides comprise one or more phosphorylation sites that are conformationally exposed to the exterior surface of the helical bundle and one or more chimeric polypeptides comprise one or more phosphorylation sites that are conformationally buried within the helical bundle such that the phosphorylation sites are not exposed.

In some aspects, a kinase phosphorylates the at least one phosphorylation site, e.g., tyrosine, serine, or threonine, on the exterior surface of the helical bundle. In some aspects, the phosphorylated site changes the confirmation of the helical bundle so that one or more phosphorylation sites, e.g., tyrosine, serine, or threonine, not exposed on the surface of the helical bundle are exposed on the surface. In some aspects, the phosphorylated sites change the confirmation of the helical bundle so that the bioactive peptide is exposed on the surface of the helical bundle.

A kinase that can phosphorylate the chimeric polypeptide of the disclosure can be naturally occurring. In some aspects, a kinase that can phosphorylate the chimeric polypeptide can be exogenously added to induce phosphorylation. In some non-limiting aspects, a kinase that can phosphorylate the chimeric polypeptide can become activated in response to a cellular stimulus (e.g. stimulation of a T cell receptor, stimulation of a B cell receptor, stimulation of a chimeric antigen receptor, activation of a G protein-coupled receptor, activation of a growth receptor, etc.). Protein kinases are known to act on proteins, by phosphorylating them on their serine, threonine, tyrosine, or histidine residues. Phosphorylation can modify the function of a protein in many ways. It can increase or decrease a protein's activity, stabilize it or mark it for destruction, localize it within a specific cellular compartment, and it can initiate or disrupt its interaction with other proteins.

In some aspects, a kinase that can phosphorylate the chimeric polypeptide of the disclosure is a Src kinase. Src kinase family is a family of non-receptor tyrosine kinases that includes nine members: Src, Yes, Fyn, and Fgr, forming the SrcA subfamily, Lck, Hck, Blk, and Lyn in the SrcB subfamily, and Frk in its own subfamily. Frk has homologs in invertebrates such as flies and worms, and Src homologs exist in organisms as diverse as unicellular choanoflagellates, but the SrcA and SrcB subfamilies are specific to vertebrates. Src family kinases contain six conserved domains: a N-terminal myristoylated segment, a SH2 domain, a SH3 domain, a linker region, a tyrosine kinase domain, and C-terminal tail.

Src family kinases interact with many cellular cytosolic, nuclear and membrane proteins, modifying these proteins by phosphorylation of tyrosine residues. A number of substrates have been discovered for these enzymes. Deregulation, including constitutive activation or over expression, may contribute to the progression of cellular transformation and oncogenic activity.

In some aspects, a kinase useful for the present disclosure includes any known kinase in the art, e.g., cyclin dependent kinases (CDKs), mitogen-activated protein kinases, etc.

### II.A. Alpha Helix

The helical bundle for the present disclosure comprises at least about 2, at least about 3, at least about 4, at least about 5, at least about 6, or at least about 7 alpha helices that interact with each other. In various embodiments, the helical bundle comprises 3-7, 4-7, 5-7, 6-7, 2-6, 3-6, 4-6, 5-6, 2-5, 3-5, 4-5, 2-4, 3-4, 2-3, 2, 3, 4, 5, 6, or 7 alpha helices. In some aspects, the helical bundle comprises about three alpha helices. In some aspects, the helical bundle comprises about four alpha helices. In some aspects, the helical bundle comprises about five alpha helices. In some aspects, the helical bundle comprises about six alpha helices. These polypeptides may be used, for example, as polypeptides that are described in more detail herein.

Alpha helix (α-helix) is a common motif in the secondary structure of proteins and is a right hand-helix conformation in which every backbone N—H group hydrogen bonds to the backbone C═O group of the amino acid located three or four residues earlier along the protein sequence.

Helices observed in proteins can range from four to over forty residues long, but a typical helix contains about ten amino acids (about three turns). In general, short polypeptides do not exhibit much α-helical structure in solution, since the entropic cost associated with the folding of the polypeptide chain is not compensated for by a sufficient amount of stabilizing interactions. Crosslinks can be incorporated into peptides to conformationally stabilize helical folds. Crosslinks stabilize the helical state by entropically destabilizing the unfolded state and by removing enthalpically stabilized “decoy” folds that compete with the fully helical state. It has been shown that α-helices are more stable, robust to mutations and designable than β-strands in natural proteins, and also in artificial designed proteins.

Since the α-helix is defined by its hydrogen bonds and backbone conformation, the most detailed experimental evidence for α-helical structure comes from atomic-resolution X-ray crystallography. Protein structures from NMR spectroscopy show helices well, with characteristic observations of nuclear Overhauser effect (NOE) couplings between atoms on adjacent helical turns. In some cases, the individual hydrogen bonds can be observed directly as a small scalar coupling in NMR.

There are several lower-resolution methods for assigning general helical structure. The NMR chemical shifts (in particular of the Cα, Cβ, and C′) and residual dipolar couplings are often characteristic of helices. The far-UV (170-250 nm) circular dichroism spectrum of helices is also idiosyncratic, exhibiting a pronounced double minimum at around 208 and 222 nm. Infrared spectroscopy is rarely used, since the α-helical spectrum resembles that of a random coil (although these might be discerned by, e.g., hydrogen-deuterium exchange). Finally, cryo electron microscopy is now capable of discerning individual α-helices within a protein, although their assignment to residues is still an active area of research.

Long homopolymers of amino acids often form helices if soluble. Such long, isolated helices can also be detected by other methods, such as dielectric relaxation, flow birefringence, and measurements of the diffusion constant. In stricter terms, these methods detect only the characteristic prolate (long cigar-like) hydrodynamic shape of a helix, or its large dipole moment.

Different amino-acid sequences have different propensities for forming α-helical structure. Methionine, alanine, leucine, glutamate, and lysine uncharged (“MALEK” in the amino-acid 1-letter codes) all have especially high helix-forming propensities, whereas proline and glycine have poor helix-forming propensities. Proline either breaks or kinks a helix, both because it cannot donate an amide hydrogen bond (having no amide hydrogen), and also because its sidechain interferes sterically with the backbone of the preceding turn—inside a helix, this forces a bend of about 30° in the helix's axis. However, proline can be seen as the first residue of a helix as it can provide structural rigidity. At the other extreme, glycine also tends to disrupt helices because its high conformational flexibility makes it entropically expensive to adopt the relatively constrained α-helical structure.

In some aspects of the present disclosure, the alpha helices of the helical bundle can be further modified to increase or decrease properties of the alpha helices. For example, an amino acid in an alpha helix can be substituted with an amino acid, e.g., glycine, such that the flexibility of the alpha helix is increased. In some aspects, the alpha helices useful for the present disclosure can be modified to increase or decrease the free energy based on the free energy per residue shown in Table 1.

TABLE 1
Standard amino acid alpha-helical propensities
Differences in free energy per residue

| | | | Helical penalty | |
|---|---|---|---|---|
| Amino acid | 3-letter | 1-letter | kcal/mol | kJ/mol |
| Alanine | Ala | A | 0.00 | 0.00 |
| Arginine | Arg | R | 0.21 | 0.88 |
| Asparagine | Asn | N | 0.65 | 2.72 |
| Aspartic acid | Asp | D | 0.69 | 2.89 |
| Cysteine | Cys | C | 0.68 | 2.85 |
| Glutamic acid | Glu | E | 0.40 | 1.67 |
| Glutamine | Gln | Q | 0.39 | 1.63 |
| Glycine | Gly | G | 1.00 | 4.18 |
| Histidine | His | H | 0.61 | 2.55 |
| Isoleucine | Ile | I | 0.41 | 1.72 |
| Leucine | Leu | L | 0.21 | 0.88 |
| Lysine | Lys | K | 0.26 | 1.09 |
| Methionine | Met | M | 0.24 | 1.00 |
| Phenylalanine | Phe | F | 0.54 | 2.26 |
| Proline | Pro | P | 3.16 | 13.22 |
| Serine | Ser | S | 0.50 | 2.09 |
| Threonine | Thr | T | 0.66 | 2.76 |
| Tryptophan | Trp | W | 0.49 | 2.05 |
| Tyrosine | Tyr | Y | 0.53 | 2.22 |
| Valine | Val | V | 0.61 | 2.55 |

In various embodiments, each helix is independently between 30-55, 30-50, 30-45, 30-40, 30-37, 33-58, 33-55, 33-50, 33-45, 33-40, or 33-37 amino acids in length. In some aspects, each helix is between 30 and 55 amino acids in length. In some aspects, each helix is between 30 and 40 amino acids in length. In some aspects, each helix is between 40 and 50 amino acids in length. In some aspects, each helix is between 35 and 45 amino acids in length. In some aspects, each helix is between 45 and 55 amino acids in length.

In some aspects, two helices in a helical bundle is linked by a linker, e.g., amino acid linker.

### II.B. Linker

The helical bundles of the present disclosure further comprise a linker. One or more linkers can be present between any two alpha helices or between an alpha helix and a bioactive peptide.

The linker useful in the present disclosure can comprise any organic molecule. In some aspects, the linker is an amino acids sequence. The linker can comprise 1-5 amino acids, 1-10 amino acids, 1-15 amino acids, or 10-15 amino acids.

In various embodiments, each amino acid linker is independently 3-10, 4-10, 5-10, 6-10, 7-10, 8-10, 9-10, 2-9, 3-9, 4-9, 5-9, 6-9, 7-9, 8-9, 2-8, 3-8, 4-8, 5-8, 6-8, 7-8, 2-7, 3-7, 4-7, 5-7, 6-7, 2-6, 3-6, 4-6, 5-6, 2-5, 3-5, 4-5, 2-4, 3-4, 2-3, 2, 3, 4, 5, 6, 7, 8, 9, or 10 amino acids in length. In all embodiments, the linkers may be structured or flexible (e.g. poly-GS).

In some aspects, the linker comprises the sequence Gₙ. The linker can comprise the sequence (GA)ₙ. The linker can comprise the sequence (GGS)ₙ. In some aspects, the linker comprises (GGGS)ₙ (SEQ ID NO:37). In some aspects, the linker comprises the sequence (GGS)ₙ(GGGGS)ₙ (SEQ ID NO:38). In these instances, n may be an integer from 1-10, i.e., 1, 2, 3, 4, 5, 6, 7, 8, 9, 10. Examples of linkers include, but are not limited to, GGG, SGGSGGS (SEQ ID NO:39), GGSGGSGGSGGSGGG (SEQ ID NO:40), GGSGGSGGGGSGGGGS (SEQ ID NO:41), GGSGGSGGSGGSGGSGGS (SEQ ID NO:42), or GGGGSGGGGSGGGGS (SEQ ID NO:43). The linker does not eliminate or diminish the alpha helix activity or the bioactive peptide. Optionally, the linker enhances the alpha helix activity or the bioactive peptide, e.g., by further providing a hydrogen bond network to the alpha helices. In some aspects, the linker for the helical bundle is (GGGGS)ₙ (SEQ ID NO:44) where G represents glycine, S represents serine and n is an integer from 1-10. In a specific embodiment, n is 3 (GGGGSGGGGSGGGGS; SEQ ID NO:45).

The linker can also incorporate a moiety capable of being cleaved either chemically (e.g., hydrolysis of an ester bond), enzymatically (i.e., incorporation of a protease cleavage sequence), or photolytically (e.g., a chromophore such as 3-amino-3-(2-nitrophenyl) proprionic acid (ANP)) in order to release one molecule from another.

In some aspects, the linker is a cleavable linker. The cleavable linkers can comprise one or more cleavage sites at the N-terminus or C-terminus or both. In some aspects, the cleavable linker consists essentially of or consists of one or more cleavable sites. In some aspects, the cleavable linker comprises heterologous amino acid linker sequences described herein or polymers and one or more cleavable sites.

In some aspects, the cleavage site is cleaved by a protease, e.g., TEV, thrombin, and/or cathepsin. Non-limiting examples of the cleavage sites are shown below:
TEV protease cleavage site: ENLYFQ(G)-X, wherein (G) can also be S, last position, −X can be anything except Proline (SEQ ID NO:46)
Thrombin protease cleavage site: LVPRGS (SEQ ID NO:47)
Cathepsin cleavage site: RLVGFE (SEQ ID NO:48)

### II.C. Bioactive Peptide

The chimeric polypeptides of the present disclosure further comprises a bioactive peptide within the helical bundle. In some aspects, a bioactive peptide can be inserted within an alpha helix in the helical bundle. In some aspects, a bioactive peptide is inserted between two alpha helices. In some aspects, the chimeric polypeptide comprises at least one, at least two, at least three, at least four, or at least five bioactive peptide. In some aspects, the bioactive peptide can be inserted or linked to one or more alpha helices via a linker. Additional disclosure for the exemplary linkers is shown elsewhere herein.

The bioactive peptide refers to an agent that has activity in a biological system (e.g., a cell or a human subject), including, but not limited to a protein, polypeptide or peptide including, but not limited to, a structural protein, an enzyme, a cytokine (such as an interferon and/or an interleukin), an antibiotic, a polyclonal or monoclonal antibody, or an effective part thereof, such as an Fv fragment, which antibody or part thereof can be natural, synthetic or humanized, a peptide hormone, a receptor, a signaling molecule or other protein; or a virus or virus-like particles. In certain aspects, a bioactive peptide comprises a therapeutic peptide or protein (e.g., protein, enzyme, antigen, or other therapeutic peptide disclosed herein), an antibody or an antigen-binding fragment thereof, an immune modulator, or any combination thereof. In some aspects, the bioactive peptide comprises a protein, an antibody, an enzyme, a peptide, or any combination thereof.

In some aspects, the bioactive peptide can be a marker protein, e.g, fluorescence peptide, e.g, GFP, luciferase, strep tag, His tag, or any combination thereof. In some aspects, the bioactive peptide is an enzyme. In some aspects, the bioactive useful for the disclosure is an epitope. Non-limiting examples of bioactive peptides useful for the present polypeptides are shown in Table 2.

TABLE 2
modified GFP-11 segment, DHMVLHERVNAAGIT (SEQ ID NO: 49)
bak bh3 peptide, GQVGRQLAIIGDDINR (SEQ ID NO: 50)
modified bak bh3 peptide, GQGGRQMAISGDDNNR (SEQ ID NO: 51)
part of the DIA peptide segment, MDAALDDLIDTLGG (SEQ ID NO: 52) from
calpastatin
VSGWRLFKKIS (SEQ ID NO: 53) - peptide for the split Luciferase NanoBit
GFP11 fluorescence peptide and binding peptide to GFP1-10:
RDHMVLHEYVNAAGIT (SEQ ID NO: 54)
BIM binding peptide and apoptotic peptide to BCL-2: IxxxLRxIGDxFxxxY
(SEQ ID NO: 55), where x is any amino acid; in one embodiment, the
peptide is EIWIAQELRRIGDEFNAYYA (SEQ ID NO: 56)
Designed peptide for binding to BCL-2:
KMAQELIDKVRAASLQINGDAFYAILRAL (SEQ ID NO: 57)
StreptagII binding peptide to streptactin or an antibody: (N)WSHPQFEK
(SEQ ID NO: 58)
EZH2 binding peptide to recruit DNA-methylases:
TMFSSNRQKILERTETLNQEWKQRRIQ (SEQ ID NO: 59)
MDM2 binding peptide to recruit p53: ETFSDLWKLL (SEQ ID NO: 60)
CPS binding peptide: GELDELVYLLDGPGYDPIHSDVVTRGGSHLFNF
(SEQ ID NO: 61)
9aaTAD1 for transcriptional activation: TMDDVYNYLFDD (SEQ ID NO: 62)
9aaTAD2 for transcriptional activation: LLTGLFVQYLFDD (SEQ ID NO: 63)
9aaTAD3 for transcriptional activation: DDAVVESFFSS (SEQ ID NO: 64)
9aaTAD4 for transcriptional activation: GDFLSDLFD (SEQ ID NO: 65)
9aaTAD5 for transcriptional activation: GDVLSDLVD (SEQ ID NO: 66)
Mad1-SID - epigenetic modification: NIQMLLEAADYLE (SEQ ID NO: 67)
Mad1-SID (3A mutant) - epigenetic modification: NIAMLLAAAAYLE
(SEQ ID NO: 68)
RHIM Domain 1 from ZBP1: IQIG (SEQ ID NO: 69)
RHIM Domain 2 from ZBP1: VQLG (SEQ ID NO: 70)
nanoBit Split Luciferase: VSGWRLFKKIS (SEQ ID NO: 71)
CC-A: GLEQEIAALEKENAALEWEIAALEQGG (SEQ ID NO: 72)
CC-B: GLKQKIAALKYKNAALKKKIAALKQGG (SEQ ID NO: 73)
GCN4: RMKQLEDKVEELLSKNYHLENEVARLKKLVGER (SEQ ID NO: 74)
CC-Di: GEIAALKQEIAALKKENAALKWEIAALKQG (SEQ ID NO: 75)
Membrane-disrupting/cell-penetrating peptides:
GALA for membrane disruption: WEAALAEALAEALAEHLAEALAEALEALAA
(SEQ ID NO: 76)
Aurein 1.2: GLFDIIKKIAESF (SEQ ID NO: 77)
Magainin-1: GIGKFLHSAGKFGKAFVGEIMKS (SEQ ID NO: 78)
Magainin-2: GIGKFLHSAKKFGKAFVGEIMNS (SEQ ID NO: 79)
Melittin: GIGAVLKVLTTGLPALISWIKRKRQQ (SEQ ID NO: 80)
Mastoparan X: INWKGIAAMAKKLL (SEQ ID NO: 81)
Cecropin A: KWKLFKKIEKVGQNIRDGIIKAGPAVAVVGQATQIAK (SEQ ID NO: 82)
Cecropin P1: SWLSKTAKKLENSAKKRISEGIAIAIQGGPR (SEQ ID NO: 83)
Citropin 1.1: GLFDVIKKVASVIGGL (SEQ ID NO: 84)
Temporin-1Lb: NFLGTLINLAKKIL (SEQ ID NO: 85)
HPV33 L2 peptide: SYFILRRRRKRFPYFFTDVRVAA (SEQ ID NO: 86)
Adenovirus pVI membrane fusion domain: AFSWGSLWSGIKNFGSTVKNY
(SEQ ID NO: 87)
Gamma-1 peptide from flock house virus: ASMWERVKSIIKSSLAAASNI
(SEQ ID NO: 88)
Poliovirus 2B pore-forming peptide:
VTSTITEKLLKNLIKIISSLVIITRNYEDTTTVLATLALLGCDASPWQWL (SEQ ID NO: 89)
Rhinovirus pore-forming peptide: IAQNPVENYIDEVLNEVLVVPNIN
(SEQ ID NO: 90)
Influenza HA2 pore-forming peptide: FLGIAEAIDIGNGWEGMEFG
(SEQ ID NO: 91)
Influenza HA2 derivative: GLFGAIAGFIENGWEGMIDG (SEQ ID NO: 92)
HA-derived INF6: GLFGAIAGFIENGWEGMIDGWYG (SEQ ID NO: 93).

### II.D. Exemplary Phospho-Switches

Some examples of the chimeric polypeptide include, but are not limited to, the constructs in shown in Table 3. In one embodiment, the optional residues may are not included in determining percent sequence identity (residues in parentheses are optional).

TABLE 3
Amino Acid Sequences

4 Tyrosine GFP-11 Phosphoswitch
(MGSSHHHHHHSSG)SMSTDLEKSVERWRELQERLVEEIERLWREALEHS
SRSKTESSVEESIKRSLDEIERVIREALERIKELIERSERL [symbol] EELDNR
EDKELGDRALEELLRLQKKLVEDLRRLQEEMNEIARRENSGSGEGL [symbol] EA
L [symbol] EKLLDHMVIHERVNAAGITDL [symbol] EKLARRMIEEG
(SEQ ID NO: 1)
4 Tyrosine DIA
MSMSTDLEKSVERWRELQERLVEEIERLWREALEHSSRSKTESSVEESIK
RSLDEIERVIREALERIKELIERSERL [symbol] EELDNREDKELGDRAAEELL
RLQKKLVEDLRRLQEEMNEIARRENSGSGEGL [symbol] EAL [symbol] KSLMDAALDDL
IDTLGGSIDL [symbol] EKLSRRMIEEG(LEHHHHHH)
(SEQ ID NO: 2)
4 Tyrosine DIA - 3 Destabilizing Mutations
MSMSTDLEKSVERWRESQERLVEEIERLWREALEHSSRSKTESSVEESIK
RSLDEIERVIREALERIKELIERSERL [symbol] EELDNREDKELGDRAAEELL
RLQKKLVEDLRRLQEEMNEIARRENSGSGEGL [symbol] EAL [symbol] KSLMDAALDDL
IDTLGGSIDL [symbol] EKLSRRMIEEG(LEHHHHHH)
(SEQ ID NO: 3)
4 Tyrosine DIA - 5 Destabilizing Mutations
MSMSTDLEKSVERWRESQERLVEEIERAWREALEHSSRSKTESSVEESIK
RSADEIERVIREALERIKELIERSERL [symbol] EELDNREDKELGDRAAEELL
RLQKKLVEDLRRLQEEMNEIARRENSGSGEGL [symbol] EAL [symbol] KSLMDAALDDL
IDTLGGSIDL [symbol] EKLSRRMIEEG(LEHHHHHH)
(SEQ ID NO: 4)

Surface Residues in the polypeptides of SEQ ID NOS:1-4: 1,3,4,7,8,11,14,15,18,19,22,26,29,30,33,36,37,39,40,41,42,45,46,49,53,5 6,57,60,64,67,68,71,75,78,79,81,82,83,84,86,87,90,91,94,98,101,102,105, 108,109,112,113,116,120,123,124,126,127,128,132,135,136,139,143,146,147, 150,154,157,158,161,165,166,167
Bold residues are Bio-active Peptide
Bold and underlined residues are Phosphorylated Tyrosines
Italicized and underlined residues are Destabilizing Mutations

>lt2_62079_10_4
(SEQ ID NO: 5)
(GSSHHHHHHSSGS)ENEELRRIIEEHLRMARRFIENIRRLIEI [symbol] EI [symbol]
EGLGSGSGREEEKSLELSKESIRLLRELLELNRRLLELFRVRESVMELLL
DILRLTTRILEEFIKIQEEILDIQRKNTSEEILRRLVEELKKIFELLIRL
FELSIDIFRKLE
>rt1_3757_5_3
(SEQ ID NO: 6)
(GSSHHHHHHSSG)SDEEFKKLLDDMSRLLIKMFKEMLDRIIDESRKMWE
RNNATIEESLDFSKKLWKEIIRILKELSDRILEKLIRELRSVDIDERRLE
ELLKMLRKLITDIFRDLLEMFERLSEELSRSGSGEGL [symbol] AEL [symbol] EKLIRD
LRKDFEKMHSDLLRRLIEKILRI
>rt1_3757_5_1
(SEQ ID NO: 7)
(GSSHHHHHHSSG)LAEELLELLVRISKEVAKILLKAVVKIVEESVRSVK
EAEESIRLSVEVWKELIKDLLRVTVKVLKEIAERLRKLNVDHRLLEELLR
ILIELSKKLLEEALEVIIRLSKELSKVGSGEGL [symbol] ESL [symbol] EELLIRLVRR
VIKRHLELLIRVVERVVRV
>st1_653275_3_2
(SEQ ID NO: 8)
(GSSHHHHHHSSG)SKEEIIRESIEIQRRLVKRIVDELRTTVEELRRMDA
RI [symbol] ESTEEINDRILKRVSEVLREALEESRKLLDRARKEVKEKKDTEEVL
KEVLELNDRILRKALDDIRKIQERVKRENSGSGSEGL [symbol] ESL [symbol] EKLIE
DIIRRLEEAVKESVRLQEELVRKT
>st1_798059_4_1
(SEQ ID NO: 9)
(GSSHHHHHHSSG)LLEELLRMQEELNKRILEMFHKLLRDLLKMLRELLK
DRLPLEELNDHSLHLLRDLLRRILEMSEETLKHILSLWHEIESIEEIFEH
LLQLHRRIFDELRKFLDHIQDRLKKLLGSGSEGL [symbol] ESL [symbol] EKLLEEIDK
MFKDILEDLLRILDHIFRRM
>lt2_13417_11_5
(SEQ ID NO: 10)
(GSSHHHHHHSSG)SLLERLEELVKHNVDLIRRILELVERAVNI [symbol] EI [symbol]
EGLGSGSDEQEELERLQRELEEVLRRLRENIDELLKLLERTQKLVVTSVL
EEILKLIEEQLRILEEALKVLKTSAEVTKRSKELGTREDEEDTLLRLVRE
ILKLVRRLVELVRELLRLAREAT
>lt2_56239_8_2
(SEQ ID NO: 11)
(GSSHHHHHHSSG)SDEEKHEDVVRKLKRLVEELLKLVRKLVEI [symbol] EI [symbol]
EGLGSGSDEQEKSRRMTEELRRMIEEAIRALEEALRLNEKSTVRVSHWAK
EEVKRILEELLEVLREALEVLEESLRVQRRSQLHEVNEKDSKELLDRVAK
LLERIVERITEIVRR [symbol] KELSDRTR
>lt2_69233_10_1
(SEQ ID NO: 12)
(GSSHHHHHHSSG)SLLEELLKIAEDQVRLVDELVKIVDRAVEI [symbol] EI [symbol]
EGLGSGSVNEKAEELQRRTKRILEELKRSAEEIEDLLRKTKKLEVHDLEE
KILDVQKKILRLVEEILRLSKRILELTRRSRVRITESLREELVRAVEELV
KVVREAVELVRRSVEIVRERT
>lt2_97000_3_3
(SEQ ID NO: 13)
(GSSHHHHHHSSG)DEKEELEKVVRKSRKLIEELLRLVRELLEI [symbol] EI [symbol]
EGLGSGSSASEDLIRINKRVLDLIEEVLESQKELVRLVEESKKHLDKRTE
EELIEDVLRKSLRVVERLLELIRRSLEIVKKSTEVLRDSTKEELLEVVRE
AVRVVEELVKIIRELVRILTETG
>rt1_2082_7_1
(SEQ ID NO: 14)
(GSSHHHHHHSS)GLLEELIKLLKKISEELVRKALKEWVKAVDENAKRVK
EEPEDHFVELSVRLSVKMIERVLRQLLEDTVQVLREIVERVVWEIDELKE
EALRVLIKISSKLVRELVKLAVKVSKELTKRVSGSEGL [symbol] EAL [symbol] ARLIS
EIVKRALEEHSELLIRIVRELVKV
>lt2_47178_5_1
(SEQ ID NO: 15)
(GSSHHHHHHSSG)REKEEQRKVVKELVRIAREAVDEVRRAVEI [symbol] EI [symbol]
EGLGSGSDKSEEALRVSEELLRKVTELLKMVEKIVDISRKSTDKDTTDRK
EDLLRVIEELLRLVRRMVEIVRELVRLSRESTHIVREDSREELVKLVTEL
VKVAEDLVRVAEE [symbol] VKISEEET
>rt1_1613_14_4
(SEQ ID NO: 16)
(GSSHHHHHHSSG)WQDEFSRMFRESSKKLIDIFERMIEEIIDRNEKIIL
VLHVEKEESLDMSQKLLEEIIELLREMQERILEEIFRAESSHDEKKEEFL
EKLRELIERTLKHFLRM [symbol] HKIIRELSERIGSGSGSEGL [symbol] AEL [symbol] SEL
SRRLLEEMMRMNTKLIEELLRELREM
>lt2_36074_13_1
(SEQ ID NO: 17)
(GSSHHHHHHSSG)EERERLLKQVDDTVKRLEEAVKRLREAVNI [symbol] EI [symbol]
EGLGSGDRSEDLLRQTREQLKTLEEVIRKLDESLKTVKKSQKKDTETDVL
EKLLEVNDRIKKVIEKLKKVLEESLRVLEKNVNNVEGREKIKEVVRILEE
LVETLEKLIKKHLDLVRKKT
>rt1_1613_15_3
(SEQ ID NO: 18)
(GSSHHHHHHSSG)ASKRLLDLVLEISKRVVENLLKLLEEVVRENAKEVR
HRSSEDSIRKSKKALEEVVREVLRQLVEVLERIVREVNVDERLKEEVLRI
AIEISERVLREAVKR [symbol] IRVSTEMSRRSGSEGL [symbol] ESL [symbol] AELVRRIVK
EVLERHSRALMEVVKRVVKL
>st1_575025_2_2
(SEQ ID NO: 19)
(GSSHHHHHHSSG)KTEEVIRKSIEEIREVVREVVELLRRVVEKNKRTMR
DERSKDEAVKRSLETAKRAIDELLKVSKKLIDDLKKTVDISEDADEIITT
LLDLNRRAVEELTRVIERIIRELKKATGSGSEGL [symbol] EAL [symbol] ERLVRELE
KILEDLVRKHVELLKKLRRDQ
>lt2_26_9_1
(SEQ ID NO: 20)
(GSSHHHHHHSSG)SEEEELLKMARKNFEMIRKMVETVKEAVNI [symbol] EI [symbol]
EGLGSGSDRSEESLRLSEESLRVIREILKLTEEALELIRRTQKKDTDDSV
MEELLRVLKEQLEVLKELLEVQEKSLKIQRESSDDRDKDSKELIKDVVEK
IERAVRLVKEVVDRSLDIAEKLR
>rt1_1613_15_4
(SEQ ID NO: 21)
(GSSHHHHHHSSG)LAEELLRLLKKSSREVVEKLLRILVELVKENVRQVT
EDKMKEKSIRKSVEVLKEVIERVLRLQVKVIEEILRRVVPDLELKEELLR
LLIEIVERTVREALRV [symbol] IEISVKASEEGSGEGL [symbol] ESL [symbol] LELVERIV
REVAKRNTEAVIEIVKRVVKM
>rt1_1613_15_5
(SEQ ID NO: 22)
(GSSHHHHHHSSG)FAEELLRLVAESSERVVRELLKLLLKAVRENVKVAT
VAEDSIEKSKRVLEKVLEDLLRRQVRMLEEIMRVVIMSDELKKEALEEII
RIIKESVERALEK [symbol] IRLSKKMSREVGSGSGSEGL [symbol] EAL [symbol] LKLVREI
VKEVVEENLRLLIEIVKEVVKV
>st1_2064_5_1
(SEQ ID NO: 23)
(GSSHHHHHHSSG)DLEELARESIELLRTIVEEIIRLIRKSADDSKRHKL
RRREITETNEEILKRSLDLQVKLLKEVLERIRRVQRDILELVRKEDVKEM
LEEVLKRVEEVIRRLLDLSRRIVERLTRENSGSGEGL [symbol] ESL [symbol] EELVK
EIVRVLEKIVRE [symbol] AELQRELIERS
>lt2_13417_10_2
(SEQ ID NO: 24)
(GSSHHHHHHSSG)SMEERLKKLLERQIRLIEELKRLVDRLEEI [symbol] EI [symbol]
EGLGSGSSLIEISEELIRMTEDLFRKLRRLLEESLKLFDDMNDTSGLLEL
LKELQHRFLRILERLLETQRTSLELQRRSVEHHVPMESIKEILHRIIRIF
KELIKILLELSRLFKHIIEHLI
>st1_653275_2_3
(SEQ ID NO: 25)
(GSSHHHHHHSSG)SLREAVKRSIEIQEDMVRRLKDILKEVADRLTKETD
ERSSDEINEKSLKDAKRILEEALRELKRLVDEIKKIESKDTEEVLRTVLE
LNKRLVEELLEDIKRVQEKVKKDGSEGL [symbol] ESL [symbol] ERLLEEIIKKLEKV
LRESAKLQREAVEKQ
>st1_569105_8_1
(SEQ ID NO: 26)
(GSSHHHHHHSSG)WLEDIFRIIIKLTEDFLRMLKELLERSLDHNKKNSR
PIEESNDTSLKLQEELLDTFLKVQEDLLDKLRRRVVREWLEELIRMFQES
MRRLIEIWKEMLTRLLEEFKRRIGSGSEGL [symbol] EAL [symbol] EELLRRLLKLFK
DLLRRQKKLLEELLKRW
>rt1_222_4_1
(SEQ ID NO: 27)
(GSSHHHHHHSSG)SKKELEDLLKRLSEKLEEMLLKLFRDLHKDNKRLVE
RKEESLEQLKKLQRDLFRHILELTKRLLEELRDRLMKNKVIVDERWIEEL
IEMLKELSERIFDKFLKMSEKLSEELSRRISGSGSGEGL [symbol] AEL [symbol] ENL
LERLIREFIKMHLRLLEELIDRIIRI
>rt1_4218_20_1
(SEQ ID NO: 28)
(GSSHHHHHHSSG)WQDELREMFKEISKILLDIFREMIKEILDRNEKLWR
HLDKEESKRILEELLREIIRILREISKRLLQRIIEILDEVNVPESSKEEF
LKMLEKILEELFRKFLEM [symbol] KRLSRKLTDSGSGEGL [symbol] SEL [symbol] EDLIRK
LEEKMIRMHTELIERFIDKLLKK
>st1_266172_2_1
(SEQ ID NO: 29)
(GSSHHHHHHSSG)SKKEMADTSIEIQKELAKRAVEVLEKVVDDLRRTGH
RKPEISEDEEEINRDSLKRIKDMLRELLREIERTLDELVRTTRKEGAPEE
TAKEIVDEVLKLNRKIVRDVLELVREAQERLTKTRGSGSGEGL [symbol] ESL [symbol]
EKLVRDIKELLRKVVEDSIRLQRDVVRRT
>lt2_91249_4_2
(SEQ ID NO: 30)
(GSSHHHHHHSSG)KDEDRLVRLAERSERLVEKAEEIVRKLAEI [symbol] EI [symbol]
EGLGSGSDEVETSLEMSRRVIELVKEAIRVVRETNELIRRSQLEIKERSE
LEELLKINEELLRLLEEWLIQKEIHRIQKESEERVSEDKKEKVVRVAKEL
ERVVREVVDIARKHAEIVKETR
>lt2_83015_5_1
(SEQ ID NO: 31)
(GSSHHHHHHSSG)DERERNVEVVREAVKTVREVLRQLEDAVEI [symbol] EI [symbol]
EGLGSGSKSEEVLKVTRKNLESIKELLKLLETVKEIQERSRTKNTEDDLL
EELVRILDRLEEVVRKLIEIRRILEIIRRSTHKVVDHRSLEEEAREVVRE
LERLVRELERIVTE [symbol] EKVVRKIG
>rt1_222_4_2
(SEQ ID NO: 32)
(GSSHHHHHHSSG)AVEELITVVIEASKRVVEELVRKLAEAVERNARRIR
HVHKEESVRQLVEIQKRVLRELLKELIKVIKKILEEVVELDEKKEELLRI
LVKLNDESLREALEMSIRLSKELSKRVGSEGL [symbol] ESL [symbol] EKLLKEVVER
VVRENVKLNKEVVERVLRL
>lt2_74221_6_1
(SEQ ID NO: 33)
(GSSHHHHHHSSG)SEDELLQDMLDKSLELIKELLKLIKELVDI [symbol] EI [symbol]
EGLGSGDESEKSEELIKRSLRFLERFEKSQRDFIRILRELIEKVTHESIL
EILEEILKISKKLLDLWKEIQKESLRIQKEIITVDILDSIREILKRLIKE
LLRIVEIIVEILKELVRIIKEIV
>st1_686178_4_1
(SEQ ID NO: 34)
(GSSHHHHHHSSG)DKERAVERWRELQERLVEEIERLWREALEHSSRSKT
ESSVEESIKRSLDEIERVIREALERIKELIERLKRDADNREDKDEILEEL
LRLQKKLVEDLRRLQEEMNEIARRENSGSGEGL [symbol] EAL [symbol] EKLLREIVE
RLRRILKESIDLLKKVVEEG
>rt1_97_6_3
(SEQ ID NO: 35)
(GSSHHHHHHSSG)FVEEILELLVRTSKRLVERVVEVLVRVIEESVRRLR
DLRSEEAVEESLKMSVEVVRRLVEELVREQVKVIKKIADVADVHERLKEE
VVRLLIKIIKETAEEIVQEIIKLSVDMSRRVGSGSGSEGL [symbol] EAL [symbol] AK
LLKELVDEIVKKNTKALLEVVKRAADV
>st1_763099_3_1
(SEQ ID NO: 36)
(GSSHHHHHHSSG)SIEELLTEILRITKEMFDELLKLLEEMLRESEKMLD
DEEDHRSLEETIRTSLHIFKRMLDEILHLHRRLHEELRKMKSTEEEWLDE
MLTDILRSFEELFNDFLRLFEKIHTDLERLSGSEGL [symbol] ESL [symbol] EELLKE
LKKLLKELLRMQEEMLKELLDRV

In some aspects, the chimeric polypeptide of the present disclosure comprises an amino acid sequence having at least 40%, 45%, 50%, 55%, 60%, 65%, 70%, 75%, 80%, 85%, 90%, 91%, 92%, 93%, 94%, 95%, 96%, 97%, 98%, 99%, or 100% sequence identity along its length to the amino acid sequence selected from the non-limiting group consisting of SEQ ID NOS:1-36, wherein the chimeric polypeptide forms a helical bundle comprising between two and seven alpha helices and a bioactive peptide, wherein one or more of the alpha helices form one or more hydrogen bonds and comprise at least one phosphorylation site and wherein the bioactive peptide is conformationally placed inside the helical bundle so that the bioactive is not activated or exposed.

In some aspects, the chimeric polypeptide comprises an amino acid sequence having at least 40%, 45%, 50%, 55%, 60%, 65%, 70%, 75%, 80%, 85%, 90%, 91%, 92%, 93%, 94%, 95%, 96%, 97%, 98%, 99%, or 100% sequence identity along its length to the amino acid sequence selected from the non-limiting group consisting of SEQ ID NOS:1-36. In some aspects, the chimeric polypeptide comprises an amino acid sequence having at least 40%, 45%, 50%, 55%, 60%, 65%, 70%, 75%, 80%, 85%, 90%, 91%, 92%, 93%, 94%, 95%, 96%, 97%, 98%, 99%, or 100% sequence identity along its length to the amino acid sequence selected from the non-limiting group consisting of SEQ ID NOs:1-36 (without the optional sequence). In some aspects, the chimeric polypeptide comprises an amino acid sequence having at least 40%, 45%, 50%, 55%, 60%, 65%, 70%, 75%, 80%, 85%, 90%, 91%, 92%, 93%, 94%, 95%, 96%, 97%, 98%, 99%, or 100% sequence identity along its length to the amino acid sequence selected from the non-limiting group consisting of SEQ ID NOs:1 to 6, e.g., SEQ ID Nos: 1, 2, 3, 4, 5, or 6 (without the optional sequence). In some aspects, the chimeric polypeptide comprises an amino acid sequence having at least 40%, 45%, 50%, 55%, 60%, 65%, 70%, 75%, 80%, 85%, 90%, 91%, 92%, 93%, 94%, 95%, 96%, 97%, 98%, 99%, or 100% sequence identity along its length to the amino acid sequence selected from the non-limiting group consisting of SEQ ID NOs:7 to 12, e.g., SEQ ID NOs: 7, 8, 9, 10, 11, or 12, (without the optional sequence). In some aspects, the chimeric polypeptide comprises an amino acid sequence having at least 40%, 45%, 50%, 55%, 60%, 65%, 70%, 75%, 80%, 85%, 90%, 91%, 92%, 93%, 94%, 95%, 96%, 97%, 98%, 99%, or 100% sequence identity along its length to the amino acid sequence selected from the non-limiting group consisting of SEQ ID NOs:13 to 18, e.g., SEQ ID NOs: 13, 14, 15, 16, 17, or 18, (without the optional sequence). In some aspects, the chimeric polypeptide comprises an amino acid sequence having at least 40%, 45%, 50%, 55%, 60%, 65%, 70%, 75%, 80%, 85%, 90%, 91%, 92%, 93%, 94%, 95%, 96%, 97%, 98%, 99%, or 100% sequence identity along its length to the amino acid sequence selected from the non-limiting group consisting of SEQ ID NOs:19 to 25, e.g., SEQ ID NOs: 19, 20, 21, 22, 23, 24, or 25 (without the optional sequence). In some aspects, the chimeric polypeptide comprises an amino acid sequence having at least 40%, 45%, 50%, 55%, 60%, 65%, 70%, 75%, 80%, 85%, 90%, 91%, 92%, 93%, 94%, 95%, 96%, 97%, 98%, 99%, or 100% sequence identity along its length to the amino acid sequence selected from the non-limiting group consisting of SEQ ID NOs:26 to 31, e.g., SEQ ID NOs: 26, 27, 28, 29, 30, or 31 (without the optional sequence). In some aspects, the chimeric polypeptide comprises an amino acid sequence having at least 40%, 45%, 50%, 55%, 60%, 65%, 70%, 75%, 80%, 85%, 90%, 91%, 92%, 93%, 94%, 95%, 96%, 97%, 98%, 99%, or 100% sequence identity along its length to the amino acid sequence selected from the non-limiting group consisting of SEQ ID NOs:32 to 36, e.g., SEQ ID NOs: 32, 33, 34, 35, or 36 (without the optional sequence).

In some aspects, the chimeric polypeptide comprises an amino acid sequence having at least 40%, 45%, 50%, 55%, 60%, 65%, 70%, 75%, 80%, 85%, 90%, 91%, 92%, 93%, 94%, 95%, 96%, 97%, 98%, 99%, or 100% sequence identity along its length to the amino acid sequence selected from the non-limiting group consisting of SEQ ID NOS:1-4, wherein no more than 2, 1, or no phosphorylation sites are present at residues corresponding to residues 1, 3, 4, 7, 8, 11, 14, 15, 18, 19, 22, 26, 29, 30, 33, 36, 37, 39, 40, 41, 42, 45, 46, 49, 53, 56, 57, 60, 64, 67, 68, 71, 75, 78, 79, 81, 82, 83, 84, 86, 87, 90, 91, 94, 98, 101, 102, 105, 108, 109, 112, 113, 116, 120, 123, 124, 126, 127, 128, 132, 135, 136, 139, 143, 146, 147, 150, 154, 157, 158, 161, 165, 166, and 167 of SEQ ID NOS:1-4.

In some aspects, the exemplary chimeric polypeptide can be further modified by substituting or mutating one or more amino acid residues with different amino acid residues. In some aspects, the chimeric polypeptide, after the modification, can have increased flexibility. In some aspects, the chimeric polypeptide, after the modification, can have decreased flexibility.

### Nucleic Acids

Another aspect the disclosure provides nucleic acids encoding the polypeptide of any embodiment or combination of embodiments of each aspect disclosed herein. The nucleic acid sequence may comprise single stranded or double stranded RNA or DNA in genomic or cDNA form, or DNA-RNA hybrids, each of which may include chemically or biochemically modified, non-natural, or derivatized nucleotide bases. Such nucleic acid sequences may comprise additional sequences useful for promoting expression and/or purification of the encoded polypeptide, including but not limited to polyA sequences, modified Kozak sequences, and sequences encoding epitope tags, export signals, and secretory signals, nuclear localization signals, and plasma membrane localization signals. It will be apparent to those of skill in the art, based on the teachings herein, what nucleic acid sequences will encode the polypeptides of the disclosure.

In a further aspect, the disclosure provides expression vectors comprising the nucleic acid of any aspect of the disclosure operatively linked to a suitable control sequence. “Expression vector” includes vectors that operatively link a nucleic acid coding region or gene to any control sequences capable of effecting expression of the gene product. “Control sequences” operably linked to the nucleic acid sequences of the disclosure are nucleic acid sequences capable of effecting the expression of the nucleic acid molecules. The control sequences need not be contiguous with the nucleic acid sequences, so long as they function to direct the expression thereof. Thus, for example, intervening untranslated yet transcribed sequences can be present between a promoter sequence and the nucleic acid sequences and the promoter sequence can still be considered “operably linked” to the coding sequence. Other such control sequences include, but are not limited to, polyadenylation signals, termination signals, and ribosome binding sites. Such expression vectors can be of any type, including but not limited plasmid, viral-based, and transposon-based expression vectors. The control sequence used to drive expression of the disclosed nucleic acid sequences in a mammalian system may be constitutive (driven by any of a variety of promoters, including but not limited to, CMV, SV40, RSV, actin, EF) or inducible (driven by any of a number of inducible promoters including, but not limited to, tetracycline, ecdysone, steroid-responsive). The expression vector must be replicable in the host organisms either as an episome or by integration into host chromosomal DNA. In various embodiments, the expression vector may comprise a plasmid, viral-based vector, or any other suitable expression vector.

Viral vectors useful for the disclosure include, but are not limited to, nucleic acid sequences from the following viruses: retrovirus, such as Moloney murine leukemia virus, Harvey murine sarcoma virus, murine mammary tumor virus, and Rous sarcoma virus; adenovirus, adeno-associated virus; SV40-type viruses; polyomaviruses; Epstein-Barr viruses; papilloma viruses; herpes virus; vaccinia virus; polio virus; and RNA virus such as a retrovirus. One can readily employ other vectors well-known in the art. Certain viral vectors are based on non-cytopathic eukaryotic viruses in which non-essential genes have been replaced with the gene of interest. Non-cytopathic viruses include retroviruses, the life cycle of which involves reverse transcription of genomic viral RNA into DNA with subsequent proviral integration into host cellular DNA. Retroviruses have been approved for human gene therapy trials. Most useful are those retroviruses that are replication-deficient (i.e., capable of directing synthesis of the desired proteins, but incapable of manufacturing an infectious particle). Such genetically altered retroviral expression vectors have general utility for the high-efficiency transduction of genes in vivo. Standard protocols for producing replication-deficient retroviruses (including the steps of incorporation of exogenous genetic material into a plasmid, transfection of a packaging cell line with plasmid, production of recombinant retroviruses by the packaging cell line, collection of viral particles from tissue culture media, and infection of the target cells with viral particles) are provided in Kriegler, M., Gene Transfer and Expression, A Laboratory Manual, W.H. Freeman Co., New York (1990) and Murry, E. J., Methods in Molecular Biology, Vol. 7, Humana Press, Inc., Cliffton, N.J. (1991).

In some aspects, the virus is an adeno-associated virus, a double-stranded DNA virus. The adeno-associated virus can be engineered to be replication-deficient and is capable of infecting a wide range of cell types and species. It further has advantages such as heat and lipid solvent stability; high transduction frequencies in cells of diverse lineages, including hemopoietic cells; and lack of superinfection inhibition thus allowing multiple series of transductions. Reportedly, the adeno-associated virus can integrate into human cellular DNA in a site-specific manner, thereby minimizing the possibility of insertional mutagenesis and variability of inserted gene expression characteristic of retroviral infection. In addition, wild-type adeno-associated virus infections have been followed in tissue culture for greater than 100 passages in the absence of selective pressure, implying that the adeno-associated virus genomic integration is a relatively stable event. The adeno-associated virus can also function in an extrachromosomal fashion.

Other vectors include plasmid vectors. Plasmid vectors have been extensively described in the art and are well-known to those of skill in the art. See, e.g., Sambrook et al., Molecular Cloning: A Laboratory Manual, Second Edition, Cold Spring Harbor Laboratory Press, 1989. In the last few years, plasmid vectors have been found to be particularly advantageous for delivering genes to cells in vivo because of their inability to replicate within and integrate into a host genome. These plasmids, however, having a promoter compatible with the host cell, can express a peptide from a gene operably encoded within the plasmid. Some commonly used plasmids available from commercial suppliers include pBR322, pUC18, pUC19, various pcDNA plasmids, pRC/CMV, various pCMV plasmids, pSV40, and pBlueScript. Additional examples of specific plasmids include pcDNA3.1, catalog number V79020; pcDNA3.1/hygro, catalog number V87020; pcDNA4/myc-His, catalog number V86320; and pBudCE4.1, catalog number V53220, all from Invitrogen (Carlsbad, Calif.). Some commonly used transposon systems include piggyBAC™, Tol2, and Sleeping Beauty™ (See, e.g., Balasubramanian et al. Comparison of three transposons for the generation of highly productive recombinant CHO cell pools and cell lines. Biotechnology and Bioengineering (2015) 113, p1234-1243.). Other plasmids are well-known to those of ordinary skill in the art. Additionally, plasmids may be custom designed using standard molecular biology techniques to remove and/or add specific fragments of DNA.

### Cells

The present disclosure provides a cell or a population of cells comprising the nucleic acid encoding the chimeric polypeptide comprising a helical bundle comprising between about two and about seven alpha-helices and a bioactive peptide, wherein one or more of the alpha helices form one or more hydrogen bonds and comprise at least one phosphorylation site and wherein the bioactive peptide is conformationally placed (i.e., buried) inside the helical bundle so that the bioactive is not activated or exposed. In some aspects, the cell or population of cells are in vitro cells. In some aspects, the cell or population of cells are in vivo cells. In some aspects, the cell or population of cells are ex vivo cells.

The expression vector or vectors can be transfected or co-transfected into a suitable target cell, which will express the polypeptides. In one aspect, the disclosure provides host cells that comprise the nucleic acids or expression vectors (i.e., episomal or chromosomally integrated) disclosed herein, wherein the host cells can be either prokaryotic or eukaryotic. The cells can be transiently or stably engineered to incorporate the expression vector of the disclosure, using techniques including but not limited to bacterial transformations, calcium phosphate co-precipitation, electroporation, or liposome mediated-, DEAE dextran mediated-, polycationic mediated-, or viral mediated transfection.

Transfection techniques known in the art include, but are not limited to, calcium phosphate precipitation (Wigler et al. (1978) Cell 14:725), electroporation (Neumann et al. (1982) EMBO J 1:841), and liposome-based reagents. A variety of host-expression vector systems may be utilized to express the proteins described herein including both prokaryotic and eukaryotic cells. These include, but are not limited to, microorganisms such as bacteria (e.g., E. coli) transformed with recombinant bacteriophage DNA or plasmid DNA expression vectors containing an appropriate coding sequence; yeast or filamentous fungi transformed with recombinant yeast or fungi expression vectors containing an appropriate coding sequence; insect cell systems infected with recombinant virus expression vectors (e.g., baculovirus) containing an appropriate coding sequence; plant cell systems infected with recombinant virus expression vectors (e.g., cauliflower mosaic virus or tobacco mosaic virus) or transformed with recombinant plasmid expression vectors (e.g., Ti plasmid) containing an appropriate coding sequence; or animal cell systems, including mammalian cells (e.g., HEK 293, CHO, Cos, HeLa, HKB11, and BHK cells).

In some aspects, the cell is a eukaryotic cell. As used herein, a eukaryotic cell refers to any animal or plant cell having a definitive nucleus. Eukaryotic cells of animals include cells of vertebrates, e.g., mammals, and cells of invertebrates, e.g., insects. Eukaryotic cells of fungi specifically can include, without limitation, yeast cells. Eukaryotic cells of plants specifically can include, without limitation, arabidopsis thaliana. A eukaryotic cell is distinct from a prokaryotic cell, e.g., bacteria.

In some aspects, the eukaryotic cell is a mammalian cell. A mammalian cell is any cell derived from a mammal. Mammalian cells specifically include, but are not limited to, mammalian cell lines. In some aspects, the mammalian cell is a human cell. In some aspects, the mammalian cell is a HEK 293 cell, which is a human embryonic kidney cell line. HEK 293 cells are available as CRL-1533 from American Type Culture Collection, Manassas, Va., and as 293-H cells, Catalog No. 11631-017 or 293-F cells, Catalog No. 11625-019 from Invitrogen (Carlsbad, Calif.). In some aspects, the mammalian cell is a PER.C6® cell, which is a human cell line derived from retina. PER.C6® cells are available from Crucell (Leiden, The Netherlands). In other embodiments, the mammalian cell is a Chinese hamster ovary (CHO) cell. CHO cells are available from American Type Culture Collection, Manassas, Va. (e.g., CHO-K1; CCL-61). In still other embodiments, the mammalian cell is a baby hamster kidney (BHK) cell. BHK cells are available from American Type Culture Collection, Manassas, Va. (e.g., CRL-1632). In some aspects, the mammalian cell is a HKB11 cell, which is a hybrid cell line of a HEK293 cell and a human B cell line. Mei et al., Mol. Biotechnol. 34(2): 165-78 (2006).

In some aspects, the cell useful for the present disclosure (e.g., in vitro, in vivo, or ex vivo cells or any host cells) is a human cell. In some aspects, the cell useful for the present disclosure (e.g., in vitro, in vivo, or ex vivo cells or any host cells) is present in a patient or derived from a patient. In some aspects, the patient-derived cell is a tumor cell, cancer cell, immune cell, leukocyte, lymphocyte, T cell, regulatory T cell, effector T cell, CD4+ effector T cell, CD8+ effector T cell, memory T cell, autoreactive T cell, exhausted T cell, natural killer T cell (NKT cells), B cell, dendritic cell, macrophage, NK cell, cardiac cell, lung cell, muscle cell, epithelial cell, pancreatic cell, skin cell, CNS cell, neuron, myocyte, skeletal muscle cell, smooth muscle cell, liver cell, kidney cell, induced pluripotent stem cell (iPSC), embryonic stem cell (ESC), and/or hematopoietic stem cell (HSC). In some aspects, the cell comprises an immune cell. In some aspects, the cell comprises a T cell. In some aspects, the cell comprises a regulatory T cell. In some aspects, the cell comprises a natural killer T cell. In some aspects, the cell comprises an NK cell. In some aspects, the cell comprises an effector T cell, e.g., a CD4+ effector T cell, and/or a CD8+ effector T cell.

In some aspects, the human cell is derived from an allogeneic donor. In some aspects, the allogeneic cell is a tumor cell, cancer cell, immune cell, leukocyte, lymphocyte, T cell, regulatory T cell, effector T cell, CD4+ effector T cell, CD8+ effector T cell, memory T cell, autoreactive T cell, exhausted T cell, natural killer T cell (NKT cells), B cell, dendritic cell, macrophage, NK cell, cardiac cell, lung cell, muscle cell, epithelial cell, pancreatic cell, skin cell, CNS cell, neuron, myocyte, skeletal muscle cell, smooth muscle cell, liver cell, kidney cell, induced pluripotent stem cell (iPSC), embryonic stem cell (ESC), and/or hematopoietic stem cell (HSC).

In some aspects, the cells are engineered to comprise one or more nucleic acids encoding the chimeric polypeptide or to express the chimeric polypeptide described herein.

### Methods of Producing Chimeric Polypeptides

A method of producing a polypeptide according to the disclosure is an additional part of the disclosure. In one embodiment, the method comprises the steps of (a) culturing a host according to this aspect of the disclosure under conditions conducive to the expression of the polypeptide, and (b) optionally, recovering the expressed polypeptide. The expressed polypeptide can be recovered from the cell free extract or recovered from the culture medium. In another embodiment, the method comprises chemically synthesizing the polypeptides.

### Methods of Designing Chimeric Polypeptides

The present disclosure is directed to a method of designing a chimeric polypeptide disclosed herein. In some aspects, the method comprises adding at least one phosphorylation site, e.g., tyrosine, serine, or threonine, in a helical bundle, which comprises about two to seven alpha helices and a bioactive peptide, wherein the at least one phosphorylation site is conformationally within the helical bundle such that the phosphorylation site is not exposed.

The present disclosure also provides a method of designing an activatable chimeric polypeptide comprising adding at least one phosphorylation site, e.g., tyrosine, serine, or threonine, in a helical bundle, which comprises about two to seven alpha helices and a bioactive peptide, wherein the at least one phosphorylation site is exposed on the surface of the helical bundle. In some aspects, the disclosure provides a method of sequestering a bioactive peptide in a chimeric polypeptide comprising adding at least one phosphorylation site in a helical bundle, which comprises about two to seven alpha helices and a bioactive peptide, wherein the at least one phosphorylation site is conformationally within the helical bundle such that the phosphorylation site is not exposed. In some aspects, the method further comprises modifying (e.g., substituting) one or more residues of the alpha helices in the helical bundle, thereby changing the properties of the alpha helices.

In some aspects, the method further comprises phosphorylating the at least one phosphorylation site. In some aspects, the phosphorylating site is selected from the group consisting of tyrosine, serine, or threonine. In some aspects, the phosphorylating site is tyrosine. The method described herein can design any chimeric polypeptides described herein.

The polypeptides, nucleic acids, expression vectors, and host cells may be used for any suitable purpose, including but not limited to those described herein.

### EXAMPLES

### Design of the Base Scaffold

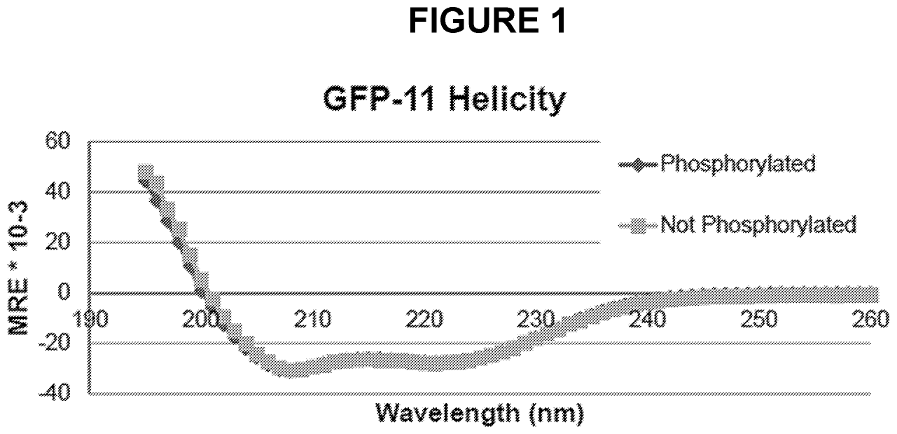

We used the Rosetta™ bundle grid sampler to generate 4 helix bundles using parametric equations. We then generated core hydrogen bond networks containing the amino acid tyrosine at “i” and “i+4” positions using HBNet from Rosetta™. This stacked tyrosine arrangement provides the most efficient arrangement of phosphorylation sites. The number of core phosphorylation sites directly relates to the amount of energy that will be can be harnessed for the destabilization of the bundle and therefore the switching function. Additionally, the tyrosine hydrogen bond networks need to remain in place after the threading of the bio-active peptide sequence to be caged. Keeping the tyrosine residues compact within the designed structure allows enough space to incorporate the bio-active peptide. We then searched and found an additional compact hydrogen bond network that contains two tyrosine residues. The tyrosine residues become Src-kinase phosphorylation sites by the addition of an “i−1” leucine. We then side chain design on the remaining residues to complete the protein. Therefore, the base scaffold is a four-helix bundle that contains 4 tyrosine hydrogen bond networks in the core which upon phosphorylation destabilize the bundle. The design was confirmed to have an alpha-helical fold by circular dichroism (CD) spectroscopy (FIG. 1).

### Phosphorylation Switch GFP-11

A modified GFP-11 segment, DHMVLHERVNAAGIT (SEQ ID NO:49), was threaded onto the sequence beginning at residue 152, creating the GFP-11 phosphorylation switch. The strand-11 of GFP peptide complements the split-GFP1-10 initiating the chromophore maturation with GFP1-10 which can then fluoresce at 508 nm after excitation at 488 nm. The GFP-11 peptide caged within the phosphorylation switch scaffold prevents the association of the GFP-11 peptide sequence when switch is not phosphorylated. After phosphorylation, the switch releases the caged peptide resulting in a large increase in fluorescence intensity.

### Phosphorylation Switch DIA

A part of the DIA peptide segment, MDAALDDLIDTLGG (SEQ ID NO:52) from calpastatin, an intrinsically disordered inhibitor of the human protease calpain, was threaded onto the phosphate switch base scaffold. The DIA peptide binds to the DIV domain of calpain, co-localizing the inhibitor the protease with nanomolar affinity. The DIA peptide caged within the scaffold prevents the association of the peptide with DIV protein. After phosphorylation, the switch releases the caged peptide resulting in switch binding to DIV.

### In Vitro Phosphorylation Reaction

50 uM of the switch was mixed with 2 mM ATP and 500 nM Src kinase domain in 10 mM HEPES pH 7.0, 10 mM magnesium chloride and 150 mM sodium chloride overnight.

### Confirming Phosphorylation via Mass Spectroscopy

We confirmed phosphorylation of the switches by electrospray ionization on a Waters Synapt G1 TOF mass spectrometer to identify the whole protein mass. Phosphorylation were identifed as (+80) mass increases dependent on the addition of kinase and ATP. Average phosphorylation was calculated by integrating the total ion current for each peak and assuming minimal changes in ionization efficiency to get the amount of each phosphorylated peak then averaging the amount of each peak.

### Methods to Detect Switching of the Phosphate Switch GFP-11

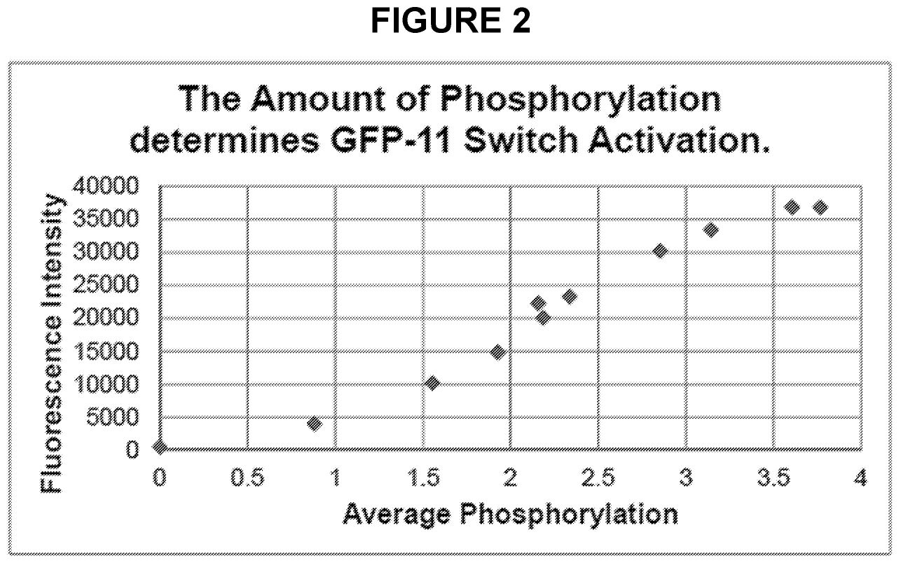

Phosphorylated or unphosphorylated GFP-11 switch (2 uM) was mixed with GFP-10 (1 uM) in 10 mM HEPES pH 7.0, 10 mM magnesium chloride and 150 mM sodium chloride. 200 uL of the mixture was put into a 96 well plate and the fluorescent intensity was read at 508 nm after excitation 488 nm on a synergy neo2 multi-mode plate reader. The plate was blanked at time zero and read again after a 48 hour incubation at room temperature. As shown in FIG. 2, the amount of phosphorylation correlates the activation of GFP fluorescence.

### Methods to Detect Switching of the Phosphate Switch DIA

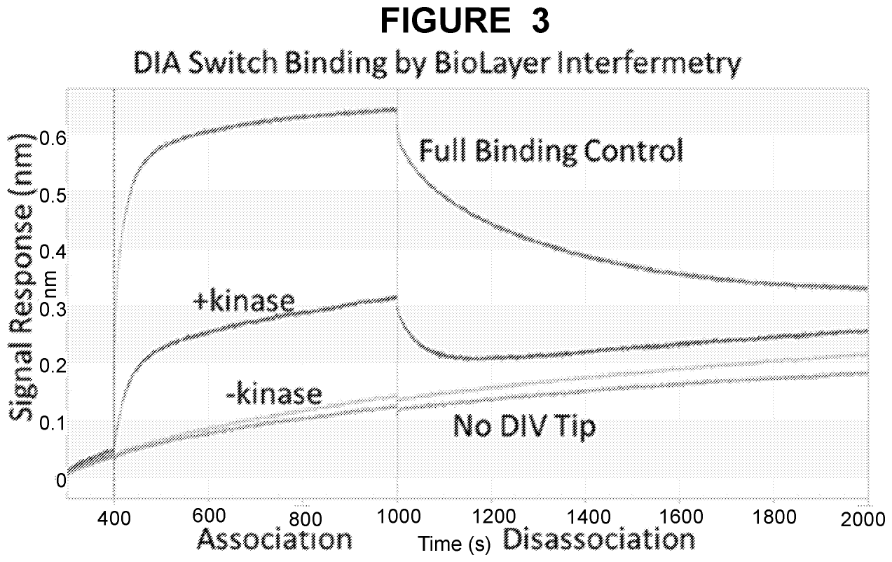

We monitored binding of the DIA calpastatin peptide to DIV domain of calpain via the Octet by ForteBio which operates by monitoring binding of the DIA switch in solution to DIV domain of calpain bound to a surface using bio-layer interferometry based on fiber optic biosensors (FIG. 3). The DIV domain protein was biotinlyated and attached to the surface via streptavidin. Phosphorylated and unphosporylated DIA switch at 2.5 uM was put into 0.5% BSA (w/v), 0.05% tween 20 (v/v), 10 mM HEPES pH 7.0, 150 mM sodium chloride and 2 mM calcium chloride and let to associate with the tip and then placed in a blank well to dissociate. The positive binding control is the GFP-11 Switch with a non-caged DIA peptide fused to the C-terminus. Since the signal response is dependent on the binding protein size, the positive controls represents maximum possible signal from this system.

### Amino Acid Sequences

Bold residues are Bio-active Peptide
Bold and underlined residues are Phosphorylated Tyrosines
Italicized and underlined residues are Destabilizing Mutations

4 Tyrosine GFP-11 Phosphoswitch
(SEQ ID NO: 1)
(MGSSHHHHHHSSG)SMSTDLEKSVERWRELQERLVEEIERLWREALEHS
SRSKTESSVEESIKRSLDEIERVIREALERIKELIERSERL [symbol] EELDNRE
DKELGDRALEELLRLQKKLVEDLRRLQEEMNEIARRENSGSGEGL [symbol] EAL [symbol]
EKLLDHMVIHERVNAAGITDL [symbol] EKLARRMIEEG
4 Tyrosine DIA
(SEQ ID NO: 2)
MSMSTDLEKSVERWRELQERLVEEIERLWREALEHSSRSKTESSVEESIK
RSLDEIERVIREALERIKELIERSERL [symbol] EELDNREDKELGDRAAEELL
RLQKKLVEDLRRLQEEMNEIARRENSGSGEGL [symbol] EAL [symbol] KSLMDAALDDL
IDTLGGSIDL [symbol] EKLSRRMIEEG(LEHHHHHH)
4 Tyrosine DIA - 3 Destabilizing Mutations
(SEQ ID NO: 3)
MSMSTDLEKSVERWRESQERLVEEIERLWREALEHSSRSKTESSVEESIK
RSLDEIERVIREALERIKELIERSERL [symbol] EELDNREDKELGDRAAEELL
RLQKKLVEDLRRLQEEMNEIARRENSGSGEGL [symbol] EAL [symbol] KSLMDAALDD
LIDTLGGSIDL [symbol] EKLSRRMIEEG(LEHHHHHH)
4 Tyrosine DIA - 5 Destabilizing Mutations
(SEQ ID NO: 4)
MSMSTDLEKSVERWRESQERLVEEIERAWREALEHSSRSKTESSVEESIK
RSADEIERVIREALERIKELIERSERL [symbol] EELDNREDKELGDRAAEELL
RLQKKLVEDLRRLQEEMNEIARRENSGSGEGL [symbol] EAL [symbol] KSLMDAALDDL
IDTLGGSIDL [symbol] EKLSRRMIEEG(LEHHHHHH)

1. A chimeric polypeptide comprising a helical bundle comprising between about two and about seven alpha-helices and a bioactive peptide, wherein one or more of the alpha helices form one or more inter-helix hydrogen bonds and comprise at least one phosphorylation site and wherein the bioactive peptide is conformationally placed inside the helical bundle so that the bioactive peptide is not activated or exposed.

2. The polypeptide of claim 1, wherein one or more of the at least one phosphorylation site is exposed to the exterior surface of the helical bundle.

3. The polypeptide of claim 1 or 2, wherein one or more of the at least one phosphorylation site is conformationally buried within the helical bundle such that the phosphorylation site is not exposed.

4. The polypeptide of any one of claims 1 to 3, wherein the at least one phosphorylation site is phosphorylated by a kinase (“phosphorylated site”).

5. The polypeptide of claim 4, wherein the phosphorylated site changes the conformation of the helical bundle and exposes one or more phosphorylation sites on the exterior surface of the helical bundle.

6. The polypeptide of any one of 4 or 5, wherein the phosphorylated site changes the conformation of the helical bundle and exposes or activates the bioactive peptide on the exterior surface of the helical bundle.

7. A chimeric polypeptide comprising a helical bundle comprising between about two and about seven alpha-helices and a bioactive peptide, wherein one or more of the alpha helices form one or more hydrogen bonds and comprise at least one phosphorylation site, wherein the phosphorylation site is phosphorylated, and wherein the bioactive peptide is conformationally exposed on the exterior surface of the helical bundle.

8. The polypeptide of any one of claims 1 to 7, wherein the helical bundle comprises at least two, at least three, at least four, or at least five phosphorylation sites.

9. The polypeptide of any one of claims 1 to 7, wherein the helical bundle comprises two, three, or four phosphorylation sites.

10. The polypeptide of any one of claims 8 to 9, wherein at least two of the phosphorylation site are separated by at least five, at least six, at least seven, at least eight, at least nine, at least ten, at least eleven, at least 12, at least 13, at least 14, at least 15, at least 16, at least 17, at least 18, at least 19, or at least 20 amino acids between the two sites.

11. The polypeptide of any one of claims 8 to 9, wherein at least two of the phosphorylation sites are separated by about two to about six amino acid residues between the two sites.

12. The polypeptide of claim 11, wherein the at least two phosphorylation sites are tyrosine residues.

13. The polypeptide of claim 12, wherein the at least two phosphorylation sites are separated by about two, about three, about four, about five, or about six amino acid residues.

14. The polypeptide of any one of claims 1 to 13, wherein the C-terminal most helical domain comprises at least one phosphorylation site.

15. The polypeptide of any one of claims 1 to 14, wherein the N-terminal most helical domain comprises at least one phosphorylation site.

16. The polypeptide of any one of claims 1 to 15, wherein at least one phosphorylation site is present on the C-terminal helix and at least one phosphorylation site is present on the N-terminal helix.

17. The polypeptide of claim 16, wherein the at least one phosphorylation site at the C-terminal helix is a tyrosine residue and the at least one phosphorylation site at the N-terminal helix is a tyrosine residue.

18. The polypeptide of any one of claims 8 to 9, wherein the at least two phosphorylation site comprises two phosphorylation sites within 2-3 amino acid residues of each other.

19. The polypeptide of claim 18, wherein each of the at least two phosphorylation sites comprises a tyrosine residue.

20. The polypeptide of any one of claims 1 to 19, further comprising an amino acid linker connecting adjacent alpha helices.

21. The polypeptide of any one of claims 1 to 20, wherein the helical bundle comprises two, three, or four alpha helices.

22. The polypeptide of any one of claims 1 to 21, wherein one or more of the at least one phosphorylation site is in the C-terminal alpha helix.

23. The polypeptide of any one of claims 1 to 21, wherein at least two or three phosphorylation sites are present on the C-terminal alpha helix and at least one phosphorylation site, such as tyrosine, is present on the N-terminal alpha helix.

24. The polypeptide of any one of claims 1 to 23, wherein each helix is independently 30 to 58 amino acids in length.

25. The polypeptide of any one of claims 20 to 24, wherein each of the amino acid linker is independently between 2 and 10 amino acids in length.

26. The polypeptide of any one of claims 1 to 25, wherein the bioactive peptide comprises comprise one or more bioactive peptide selected from Table 2.

27. The polypeptide of any one of claims 1 to 26, wherein one or more of the phosphorylation site are selected from the group consisting of tyrosine, serine, and threonine.

28. The polypeptide of claim 27, wherein the phosphorylation site is tyrosine.

29. The polypeptide of any one of claims 1 to 28, comprising an amino acid sequence having at least 40%, 45%, 50%, 55%, 60%, 65%, 70%, 75%, 80%, 85%, 90%, 91%, 92%, 93%, 94%, 95%, 96%, 97%, 98%, 99%, or 100% sequence identity along its length to the amino acid sequence selected from the non-limiting group consisting of SEQ ID NOS:1-36.

30. A chimeric polypeptide comprising an amino acid sequence having at least 40%, 45%, 50%, 55%, 60%, 65%, 70%, 75%, 80%, 85%, 90%, 91%, 92%, 93%, 94%, 95%, 96%, 97%, 98%, 99%, or 100% sequence identity along its length to the amino acid sequence selected from the non-limiting group consisting of SEQ ID NOS:1-36.

31. A chimeric polypeptide comprising an amino acid sequence having at least 40%, 45%, 50%, 55%, 60%, 65%, 70%, 75%, 80%, 85%, 90%, 91%, 92%, 93%, 94%, 95%, 96%, 97%, 98%, 99%, or 100% sequence identity along its length to the amino acid sequence selected from the non-limiting group consisting of SEQ ID NOS:1-4, wherein no more than 2, 1, or no phosphorylation sites are present at residues corresponding to residues 1,3,4,7,8,11,14,15,18,19,22,26,29,30,33,36,37,39,40,41,42,45,46,49,53,56,57,60,64,67,68,71, 75,78,79,81,82,83,84,86,87,90,91,94,98,101,102,105,108,109,112,113,116,120,123,124,126, 127,128,132,135,136,139,143,146,147,150,154,157,158,161,165,166,167 of SEQ ID NOS:1-4.

32. A plurality of chimeric polypeptides the chimeric polypeptide of any one of claims 2 and 4 to 31 and the chimeric polypeptide of any one of claims 3 to 31 in equilibrium.

33. The plurality of chimeric polypeptides of claim 32, wherein a kinase phosphorylates the at least one phosphorylation site on the surface of the helical bundle.

34. The plurality of chimeric polypeptides of claim 32 or 33, wherein the phosphorylated site changes the conformation of the helical bundle so that one or more phosphorylation sites not exposed on the surface of the helical bundle are exposed on the surface.

35. The plurality of chimeric polypeptides of any one of claims 32 to 34, wherein the phosphorylated sites change the conformation of the helical bundle so that the bioactive peptide is exposed on the surface of the helical bundle.

36. A pharmaceutical composition comprising the chimeric polypeptide of any one of claims 1 to 31 or the plurality of chimeric polypeptides of any one of claims 32 to 35.

37. A nucleic acid encoding the polypeptide of any one of claims 1 to 31 or the plurality of chimeric polypeptides of any one of claims 32 to 35.

38. An expression vector comprising the nucleic acid of claim 37 operatively linked to a regulatory sequence.

39. The vector of claim 38, which is a adenoviral vector, a lentiviral vector, a baculoviral vector, an Epstein Barr viral vector, a papovaviral vector, a vaccinia viral vector, a herpes simplex viral vector, an adeno associated virus (AAV) vector, or a transposon vector.

40. An in vitro cell comprising the nucleic acid of claim 37 or the expression vector of claim 38 or 39.

41. An ex vivo cell comprising the nucleic acid of claim 37 or the expression vector of claim 38 or 39.

42. An in vivo cell comprising the nucleic acid of claim 37 or the expression vector of claim 38 or 39.

43. A host cell comprising the nucleic acid of claim 37 or the expression vector of claim 38 or 39.

44. The cell of any one of claims 40 to 43, wherein the nucleic acid or the expression vector is integrated into a host cell chromosome.

45. The cell of any one of claims 40 to 43, wherein the nucleic acid or the expression vector is episomal.

46. The cell of any one of claims 40 to 45, which comprises mammalian cells.

47. The cell of claim 46, which comprises HEK 293, CHO, Cos, HeLa, HKB11, or BHK cells.

48. The cell of any one of claims 40 to 45, which comprises a tumor cell, cancer cell, immune cell, leukocyte, lymphocyte, T cell, regulatory T cell, effector T cell, CD4+ effector T cell, CD8+ effector T cell, memory T cell, autoreactive T cell, exhausted T cell, natural killer T cell (NKT cells), B cell, dendritic cell, macrophage, NK cell, cardiac cell, lung cell, muscle cell, epithelial cell, pancreatic cell, skin cell, CNS cell, neuron, myocyte, skeletal muscle cell, smooth muscle cell, liver cell, kidney cell, induced pluripotent stem cell (iPSC), embryonic stem cell (ESC), hematopoietic stem cell (HSC).

49. A method of designing an activatable chimeric polypeptide comprising adding at least one phosphorylation site in a helical bundle, which comprises about two to seven alpha helices and a bioactive peptide, wherein the at least one phosphorylation site is conformationally within the helical bundle such that the phosphorylation site is not exposed.

50. A method of designing an activatable chimeric polypeptide comprising adding at least one phosphorylation site in a helical bundle, which comprises about two to seven alpha helices and a bioactive peptide, wherein the at least one phosphorylation site is exposed on the surface of the helical bundle.

51. A method of sequestering a bioactive peptide in a chimeric polypeptide comprising adding at least one phosphorylation site in a helical bundle, which comprises about two to seven alpha helices and a bioactive peptide, wherein the at least one phosphorylation site is conformationally within the helical bundle such that the phosphorylation site is not exposed.

52. The method of any one of claims 49 to 51, wherein the chimeric polypeptide comprises the polypeptide of any one of claims 1 to 31.

53. The method of any one of claims 49 to 52, wherein the at least one phosphorylation site is phosphorylated by a kinase.

54. The method of any one of claims 49 to 53, further comprising phosphorylating the at least one phosphorylation site.

55. The method of any one of claims 49 to 54, wherein the phosphorylation site is selected from the group consisting of tyrosine, serine, or threonine.

56. The method of claim 55, wherein the phosphorylation site is tyrosine.

57. A method of expressing a chimeric polypeptide comprising culturing the cell of any one of claims 40 to 48 under suitable conditions.

58. The method of claim 57, wherein the cell is a mammalian cell.

59. The method of claim 57, wherein the cell comprises HEK293, CHO, Cos, HeLa, HKB11, or BHK cells.

60. The method of claim 57, wherein the cell comprises a tumor cell, cancer cell, immune cell, leukocyte, lymphocyte, T cell, regulatory T cell, effector T cell, CD4+ effector T cell, CD8+ effector T cell, memory T cell, autoreactive T cell, exhausted T cell, natural killer T cell (NKT cells), B cell, dendritic cell, macrophage, NK cell, cardiac cell, lung cell, muscle cell, epithelial cell, pancreatic cell, skin cell, CNS cell, neuron, myocyte, skeletal muscle cell, smooth muscle cell, liver cell, kidney cell, induced pluripotent stem cell (iPSC), embryonic stem cell (ESC), hematopoietic stem cell (HSC).
